SEQUENCE LISTING (5 sequences)

SEQ ID NO: 1 - DNA, 21 bp, Homo sapiens
cctcagtctggggctttcttt

SEQ ID NO: 2 - DNA, 21 bp, Homo sapiens
cttgtttgcgtccatgtggtc

SEQ ID NO: 3 - DNA, 22 bp, Homo sapiens
agttgaggggactttcccaggc

SEQ ID NO: 4 - DNA, 21 bp, Homo sapiens
gaccccactccctgctctaac

SEQ ID NO: 5 - DNA, 21 bp, Homo sapiens
agaagaggggaaggacgggag

(19) United States
(12) Patent Application Publication
(10) Pub. No.: US 2006/0165675 A1
(43) Pub. Date: Jul. 27, 2006

(54) Modulation of mesenchymal cells via iga-receptors

(76) Inventors: Janice Beth Yeved Richman-Eisenstat, Winnipeg (CA); Jing Yu, Winnipeg (CA)

Correspondence Address: BERESKIN AND PARR, 40 KING STREET WEST, BOX 401, TORONTO, ON, M5H 3Y2, CA

(21) Appl. No.: 10/520,284

(22) PCT Filed: Jul. 20, 2003
(86) PCT No.: PCT/CA03/01090

§ 371 Date: Oct. 11, 2005

Related U.S. Application Data
(60) Provisional application No. 60/400,036, filed on Aug. 2, 2002.
(60) Provisional application No. 60/400,129, filed on Aug. 2, 2002.

(51) Int. Cl. A61K 39/395

(52) U.S. Cl. 424/131.1

(57) ABSTRACT

IgA receptors, including a polymeric immunoglobulin receptor (pIgR) and a FcαR, have been found on smooth muscle cells, synovial fibroblast cells and on both synovial and endothelial cells in synovial tissues from patients with arthritis. Incubation of smooth muscle cells or tissue with pIgA increases cytosolic calcium and alters the contractile state. Incubation of synovial cells with IgA modulates the inflammatory responses of these cells. The invention relates to methods of modulating calcium signalling and/or contractility of mesenchymal cells, as well as modulating (preferably inhibiting) the inflammatory responses of mesenchymal cells, methods of treating inflammatory conditions (such as asthma and arthritis), methods of drug delivery to mesenchymal cells and methods of detecting conditions associated with IgA receptors on mesenchymal cells.

### FIELD OF THE INVENTION

The invention relates to methods of modulating mesenchymal cell biology, including cytosolic calcium signalling in and inflammatory responses of mesenchymal cells, methods of treating arthritis and asthma and methods of drug delivery to mesenchymal cells, as well as methods to diagnose IgA-receptor-mediated mesenchymal inflammation.

### BACKGROUND OF THE INVENTION

Rheumatoid Arthritis

Rheumatoid arthritis (RA) is a common chronic inflammatory and autoimmune disorder of unknown etiology that attacks adults and children (Choy and Panayi, 2001). Patients with RA have a poor long-term prognosis, with 80% becoming disabled after 20 years (Scott et al, 1987). Current treatments do not improve this prognosis. The prevalence of RA is about 1.3% and has been steadily rising. Insights into the cell biology of this disorder will go far to developing improved treatments that prevent disability and improve long-term prognosis.

RA is characterized by synovial inflammation, proliferation and progressive joint destruction (reviewed in Jenkins et al, 2002). The immune reaction begins in the synovial lining of the joint, with lymphocytes playing a significant role in acute disease and a lesser role in chronic disease. The earliest pathologic changes in the disease are microvascular injury and increased vascular permeability, accompanied by an influx of inflammatory cells (CD4 lymphocytes, neutrophils, and plasma cells) in the perivascular space. Cytokines, lymphokines, and chemokines are released. TNFα, IL-1 and IL-6 are the key cytokines that drive inflammation in RA. Patients develop swelling, pain, and joint stiffness with the onset of vascular injury and angiogenesis in the synovial membrane. Synovial proliferation and the evolving inflammation exacerbate these symptoms and progressively limit joint motion. Neutrophils accumulate in the synovial fluid in response to local production of IL-8. B lymphocytes mature into plasma cells which locally produce rheumatoid factor and other antibodies that further aggravate the inflammation. Immune complexes activate the complement system, releasing chemokines and increasing vascular permeability. Immune complexes also promote phagocytosis, leading to greater lysosomal enzyme release and the digestion of collagen, cartilage matrix, and elastic tissues. The release of oxygen free radicals injures cells, which release phospholipids that fuel the arachidonic acid cascade and exacerbate the local inflammatory response. Proliferating synovium forms an invasive pannus, eroding through cartilage and subchondral bone. Of the two types of synoviocytes, type A monocyte-like and type B fibroblast-like, the type B fibroblast-like cells stimulate the cartilage and bone destruction of chronic disease. Chondrocytes release their own proteases and collagenases, and further contribute to this self-perpetuating local immune response.

The initial inciting factor and the precise mechanism of these complex cellular interactions remain unknown. However, it is clear that RA is characterized by increased activity of the pro-inflammatory transcription factor, NFκB, in synovial fibroblasts. NFκB stimulates production of cytokines and adhesion molecules, including TNF-α, IL-1β, IL-6, IL-8 and ICAM-1.

Patients with RA may also develop systemic vasculitis, neurologic, pulmonary, cardiac and/or liver abnormalities. The number and severity of the extra-articular features vary with the duration and severity of disease. Extra-articular complications are seen in patients with high titers of rheumatoid factor (RF). RF is an immunoglobulin that binds other immunoglobulins at their Fc components, forming immune complexes. RF may consist of IgM, IgA, IgE and/or IgG isotypes, and may be found in several other diseases including Sjogren's syndrome, subacute bacterial endocarditis, mixed cryoglobulinemia, systemic lupus erythematosis, scleroderma, sarcoidosis, idiopathic pulmonary fibrosis and malignancies. However, the combined elevation of IgM-RF and IgA-RF is highly specific for RA and is very rarely found in rheumatic diseases other than RA (Jonsson et al, 1998). In a cross-sectional study, the majority (74%) of RA patients had elevations of 2-3 RF isotypes, and 67% had the combined elevation of IgA and IgM (Jonsson and Valdimarsson, 1992). Of those patients with RA, 65% are positive for IgA-RF and 92% are positive for IgM-RF (Gioud-Paquet et al, 1987).

IgA-RF can occur in serum and synovial fluid, and is predominantly polymeric (Otten et al, 1991; Schrohenloher et al, 1986). Several studies have reported significant clinical implications to IgA-RF in RA. RA patients with a predominant increase in IgA-RF have more erosive disease (Jorgensen et al, 1996). IgA-RF Is associated with extra-articular manifestations of RA (Jonsson et al, 1995; Pai et al, 1998). Detection of IgA-RF early in disease predicts poorer prognosis with a more rapidly progressive course (Teitsson et al, 1984; Pai et al, 1998; Houssien et al, 1997).

Asthma

Asthma is a chronic inflammatory airway disease with symptoms of wheezing, cough, shortness of breath and chest tightness. These symptoms vary from person to person and occur with no set pattern. The cause of asthma is not known and there is no cure. Asthmatics respond diversely to available treatments. Asthma triggers also vary from person to person. Viral respiratory infections play an important role in asthma, triggering an asthma attack and sensitizing patients to other triggers, such as dust mites, animals, molds, pollens and air pollutants (Message and Johnston, 2002; Tuffaha et al, 2000). Other symptom triggers include smoke, exercise, cold air, increased humidity, strong-smelling substances (e.g. perfume, cigarette smoke, paint fumes, dusts), sinus infection, gastric aspiration or gastroesophageal reflux, and certain food additives. Not all asthmatics are alike in their responses to these different stimuli. Some people react immediately to a stimulus; others have delayed bronchial constriction. Furthermore, different patients respond differently to currently available medications for asthma. Regardless of the heterogeneity of asthma, bronchial hyper-reactivity is its hallmark and consists of smooth muscle contraction along the bronchial tree.

A number of studies indicate a major role for alterations in the smooth muscle. Airway smooth muscle (ASM) cells exhibit a contractile phenotype and a proliferative-synthetic phenotype, capable of producing proinflammatory cytokines, chemokines and growth factors (Halayko et al, 1996; Halayko et al, 1999; Schmidt and Rabe, 2000). It is now being suggested that the ASM itself can contribute directly to the persistence of inflammation and airway remodeling that occurs in asthma. The features of this airway remodeling include epithelial damage, deposition of extracellular matrix proteins throughout the airways, goblet cell metaplasia, and smooth muscle hypertrophy (Davies et al, 2003; Holgate et al, 2000). It is unknown whether this remodeling is due to or occurs in parallel with the inflammatory response.

Little is known about the biology of IgA in asthma. However, upper airway infections are well known to frequently exacerbate the airflow obstruction that occurs in patients with asthma and other lung diseases—a situation where the concentration of immunoglobulins in the airways increases. If ASM indeed possess receptors for IgA that alter ASM biology or function, then a new pathophysiological explanation arises for these infectious exacerbations of asthma. In addition, the presence or absence of such a receptor on ASM might account, at least in part, for the variety of clinical manifestations and therapeutic responsiveness amongst patients with asthma. Furthermore, a better understanding of the cell and molecular biology of such receptors and their biology in ASM could lead to a novel approach for the diagnosis and treatment of asthma.

Immunoglobulin A (IgA) abundantly coats the enormous surface area of the mucosal epithelium, which measures about 300-400 m² in adult humans. Total IgA transport is roughly 5-15 gms per day in an adult human with 15% going into airway secretions (Childers et al, 1989). In fact, IgA comprises 5-10% of the total protein in bronchoalveolar lavage fluid (Bell et al 1981). Despite its abundance, relatively little is known about the mechanism of action of IgA in host immune defense and immune tolerance. The mucosal epithelium is physically vulnerable to continuous exposure to potentially infectious agents, such as bacteria, viruses, fungi and parasites, as well as to substances in the environment or diet. IgA is one of the most important proteins protecting the mucosal epithelium that guards the internal environment from the outside world. Elevated concentrations of IgA have been identified in induced sputum from asthmatics in contrast to that from healthy people (Louis et al, 1997; Nahm and Park, 1997; Nahm et al, 1998). Furthermore, increased levels of specific IgA antibodies to both allergen and bacterial antigen have been measured in induced sputum from asthmatics (Nahm et al, 1998).

Infections of the upper airway stimulate submucosal B cells to increase production of specific pIgA. Furthermore, if infectious pathogens resulted in the breakdown of the epithelial barrier, then cells in the subepithelial layers, such as those of ASM, become exposed to increased concentrations of pIgA as well as sIgA. Although airway inflammation and bronchoconstriction involve multifactorial and complex processes, if ASM possess receptors for IgA that are activated by different isoforms of IgA, then a novel pathophysiological mechanism is proposed to account for (1) the deterioration in airflow obstruction during infectious exacerbations in patients with asthma, (2) the induction of asthma in predisposed individuals, and (3) the temporary development of bronchial hyperreactivity in non-asthmatics (i.e. reactive airways dysfunction syndrome).

Polymeric Immunoglobulin Receptor

IgA exists in different isoforms (Mestecky et al, 1999). B lymphocytes residing in submucosal tissues produce similar proportions of polymeric IgA1 and IgA2 subclasses, secreting at least two IgA molecules linked together by a J chain. Epithelial cells of the respiratory and gastrointestinal tracts abundantly express the polymeric immunoglobulin receptor (pIgR) which transfers polymeric IgA from the submucosa (the basolateral surface of the epithelium) to the lumenal (apical) surface (Mostov et al, 1995; Mostov and Kaetzel, 1999). At the apical surface, proteolytic cleavage of the pIgR releases secretory component (SC) bound to pIgA into mucosal secretions, called secretory IgA (sIgA). SC stabilizes sIgA from proteolytic degradation by bacterial enzymes and helps neutralize pathogens, especially viruses. sIgA in mucosal secretions is the first line of defense, acting to bind microorganisms and thereby limiting adhesion and colonization. IgA may neutralize viruses and bacterial toxins by binding to antigenic determinants important in the microorganism's interaction with cellular receptors. Additional roles for sIgA are postulated to include transport of immune complexes out through the epithelial surface by the pIgR. IgA is therefore a very important first line of host immune defense at mucosal surfaces.

In contrast to mucosal secretions where sIgA prevails, the predominant form of IgA in human serum is monomeric IgA (mIgA) from B lymphocytes in the bone marrow and spleen. While the pIgR will selectively mediate transport of polymeric IgA across epithelial cells, this receptor does not bind monomeric IgA. IgA present in secretions therefore differs in biochemical properties from IgA found in serum. The polymerization state and the presence of SC might be expected to result in unique effector functions for different forms of IgA depending on the site of production and intended point of action.

Binding of pIgA to the pIgR on mucosal epithelial cells occurs via the J-chain, which covalently links the IgA molecules together as dimers and multimers (Johansen et al, 2001). In addition, pIgA, which is heavily glycosylated, can bind to asialoglycoprotein receptors on liver cells. In contrast, binding to white blood cells (neutrophils, eosinophils, monocytes/macrophages) occurs by attachment of the Fc portion of IgA to FcαR (also known as CD89) expressed on these cells.

Human pIgR can also transport polymeric IgM, which contains J-chain and increases in concentration during times of acute infections.

Fc-Alpha Receptors for IgA

Binding to white blood cells (neutrophils, eosinophils, monocytes/macrophages) occurs by attachment of the Fc portion of IgA to Fc-alpha receptors (FcαR; also known as CD89) expressed on these cells (Kerr and Woof, 1999; Morton et al, 1996). Neutrophils and monocytes/macrophages constitutively express FcαR as a 55-75 kd protein, while eosinophils express FcαR as a 70-100 kd protein with increased glycosylation (Albrechtsen et al, 1988; Monteiro et al, 1990). FcαR expression on monocytes and neutrophils increases in response to TNF-α, IL-1, GM-CSF, LPS or phorbol esters; IFN-γ and TGF-β1 decrease expression (discussed in Deo et al, 1998). The gene for FcαR is located on chromosome 19 and encodes several alternatively spliced isoforms of the receptor's α-chain (55-110 kD; Morton et al, 1996). FcαR can trigger release of inflammatory mediators and phagocytosis of IgA-coated particles (Yeaman and Kerr, 1987; Patry et el, 1995). IgA-coated neutrophils and macrophages phagocytose particles, bacteria and immune complexes more efficiently than uncoated cells. Although the concentration of the predominantly monomeric IgA in blood is high enough to completely saturate the thousands of FcαR on neutrophils, mIgA will not trigger signal transduction in PMNs unless the receptors are crosslinked (Stewart et al., 1994). The pIgA and sIgA have the potential to crosslink FcαR on cell surfaces due to their polymeric composition. So, during times of infection when submucosal B cells are stimulated to increase production of specific pIgA, myeloid cells recruited to sites of inflammation are better prepared for their functions in the mucosal lumen. FcαR-induced calcium release and subsequent cytokine production depend on association with the FcR γ-chain (Morton et al, 1995). In vivo studies in transgenic mice show that while FcR γ chain is important for FcαR-triggered phagocytosis, CR3 (CD11b/CD18) is required for FcαR-mediated antibody-dependent cellular cytotoxicity (van Egmond et al, 1999).

FcαR may play a role in cancer in addition to its function against microbial pathogens: IgA antitumor antibodies or bispecific antibodies directed to FcαR and tumor antigens effectively lyse tumor cells (Deo et al, 1998). Deo's work and that of others highlight FcαR as a potential immunotherapeutic target of malignant and infectious diseases (Valerius et al, 1997). The novel finding of the FcαR on mesenchymal cells, including synovial fibroblasts and airway smooth muscle cells, thus indicates that targeting this receptor would be a promising and novel therapeutic approach for inflammatory diseases, such as arthritis and asthma.

### SUMMARY OF THE INVENTION

The inventors have unexpectedly found that the polymeric immunoglobulin receptor (pIgR) and the Fc alpha receptor are expressed in human airway smooth muscle (ASM) cell cultures. The inventors have also shown that incubation of ASM with the ligand for pIgR (pIgA) causes a rise in intracellular calcium concentrations that is unique in that the response is delayed, sustained, oscillates and increases the sensitivity of ASM to subsequent stimulation with histamine. Incubation with mIgA (which does not bind pIgR) does not cause this effect on intracellular calcium concentrations in ASM. Smooth muscle responses to IgA have never been described before.

The inventors have also found that the Fc alpha receptor (FcαR) is expressed on synovial fibroblasts from patients with rheumatoid arthritis (RA) and osteoarthritis (OA). Furthermore, the inventors have shown that incubating synovial fibroblasts with IgA causes an increase in NFκB DNA binding, and enhances TNF-alpha-induced ICAM-1 protein expression, and TNFα-induced IL-8 and RANTES mRNA expression. As a result, inhibiting signalling through IgA receptors may be an effective means of treating arthritis and other inflammatory diseases.

Fibroblasts, smooth muscle cells and endothelial cells are mesenchymal cells. The inventors are the first to show the presence of IgA receptors on these mesenchymal cells. Modulating these receptors are useful for altering physiological responses in mesenchymal cells.

Accordingly, the present invention provides a method of modulating the inflammatory responses of a mesenchymal cell comprising administering to a cell or animal in need thereof an effective amount of an agent that can modulate an IgA receptor on a mesenchymal cell.

In a further embodiment, the present invention provides a method of treating an inflammatory condition caused by IgA binding to an IgA receptor on a mesenchymal cell comprising administering an effective amount of an IgA receptor antagonist to a patient in need thereof. In one embodiment, the present invention provides a method of treating a patient with arthritis comprising administering an effective amount of an IgA receptor antagonist to a patient in need thereof. In another embodiment, the present invention provides a method of treating a patient with asthma comprising administering an effective amount of an IgA receptor antagonist to a patient in need thereof.

The binding of IgA to an IgA receptor is known to induce cytosolic calcium signalling and cause a number of calcium dependent effects. Accordingly, the present invention also provides a method of modulating intracellular calcium signalling in a mesenchymal cell comprising administering to a cell or animal in need thereof an effective amount of an agent that can modulate an IgA receptor on a mesenchymal cell. In one embodiment, the present invention provides a method of inhibiting cytosolic calcium signalling in a mesenchymal cell comprising administering an effective amount of an IgA receptor antagonist to a cell or animal in need thereof.

The discovery of IgA receptors on mesenchymal cells allows the development of methods to target delivery of a compound or substance to a mesenchymal cell. Accordingly, the present invention also includes a method of delivering a substance to a mesenchymal cell comprising administering an effective amount of a conjugate comprising the substance coupled to an IgA receptor ligand to an animal or cell in need thereof.

The discovery of IgA receptors on synovial fibroblasts allows development of diagnostic assays to detect IgA receptor-mediated diseases or inflammatory conditions including arthritis (including rheumatoid arthritis, osteoarthritis, spondyloarthropathies) and asthma, as well as other inflammatory diseases such as Crohn's disease, Ulcerative colitis, Behcet's disease, Sjogren's disease and vasculitides.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows immunofluorescence staining for FcαR in primary cell cultures of both RA and OA synovial fibroblasts FIG. 2 shows RT-PCR product bands for IgA-binding domain of FcαR in both RA and OA synovial fibroblasts.

FIG. 3 shows immunohistochemical staining of FcαR in synovial cells and endothelial cells in human RA and OA synovial tissue samples.

FIG. 4 shows a dose-dependent increase in NFκB activity in both RA and OA synovial fibroblasts treated with increasing concentrations of pIgA.

FIG. 5A is a graph showing that IgA increases TNFα-induced ICAM-1 protein expression on RA synovial fibroblasts by cell ELISA.

FIG. 5B is a graph showing that an immune complex consisting of pIgA and an antibody to the alpha chain of IgA increases baseline RA synovial fibroblast expression of ICAM-1, in contrast to either reagent alone.

FIG. 6 is a graph showing that IgA further increases TNF-alpha induced gene expression in rheumatoid arthritis synovial fibroblasts by RNase protection assay.

FIG. 7 is a graph showing that IgA further increases TNF-alpha induced gene expression in osteoarthritis synovial fibroblasts by RNase protection assay.

FIG. 8A shows immunofluorescence staining for pIgR protein in serum-starved ASM using a rabbit antibody to human secretory component.

FIG. 8B shows immunofluorescence staining for the alpha chain of IgA in serum-starved ASM pre-incubated overnight with pIgA (live-cell uptake) in contrast to pre-incubation with mIgA.

FIG. 8C shows live uptake staining for the rabbit IgG antibody to human secretory component in contrast to pre-incubation of live ASM cells with an irrelevant rabbit IgG.

FIG. 9 shows immunofluorescence staining for FcαR in non-starving ASM cells (upper panel) and that this staining is enhanced by pre-incubating live cells with either pIgA (middle panel) or mIgA (lower panel).

FIG. 10 shows a western blot for pIgR protein in non-starving human ASM that is still present after 6 days and 12 days of serum starvation (upper panel). This blot was stripped and re-probed with a phosphotyrosine antibody to show that the pIgR protein in ASM is less phosphorylated with serum deprivation.

FIGS. 11A show a pIgR band in serum-starved cells by RT-PCR using mRNA from ASM obtained from one human subject. MDCK cells transfected with human pIgR were used as a positive control. This figure also shows FcαR mRNA in serum-fed ASM and U937 cells used as a positive control cell line, in contrast to serum-starved ASM.

FIG. 11B shows the presence of pIgR mRNA in serum-starved and serum-fed ASM obtained from a second subject by RT-PCR. FcαR mRNA is present in serum-fed ASM as determined by RT-PCR.

FIG. 12A shows calcium imaging studies in Fura-2 loaded serum-starved ASM stimulated with 12 μg/ml pIgA. At 80 min, pIgA causes an initial rise in cytosolic calcium concentrations and this effect becomes sustained at 110 min. At this point, the calcium concentrations oscillate. The oscillations abate and concentrations decrease 10 min after washing off the pIgA.

FIG. 12B shows calcium imaging studies in Fura-2 loaded serum-starved ASM in buffer alone (time course study).

FIG. 13A shows calcium imaging studies in Fura-2 loaded serum-starved ASM pre-stimulated with histamine which is washed off prior to adding low dose pIgA (0.12 μg/ml). Cytosolic calcium concentrations begin to rise after 1 h and increase 4-6 fold with histamine stimulation after washing off pIgA.

FIG. 13B shows calcium imaging studies in Fura-2 loaded serum-starved ASM incubated with buffer alone (time control) after pre-stimulation with histamine. These cells were re-exposed to histamine after 2 hours in buffer and showed less than a 2-fold increase in cytosolic calcium concentrations.

FIG. 13C shows calcium imaging studies in Fura-2 loaded serum-starved ASM stimulated with mIgA following pre-stimulation with histamine. (These serum-starved ASM do not express FcαR making mIgA a negative control.) After washing off mIgA, the responses to histamine are augmented by a factor of less than 2, as in FIG. 13B with buffer alone.

FIG. 13D shows calcium imaging studies in Fura-2 loaded serum- starved ASM stimulated with high dose pIgA 12 μg/ml following stimulation with histamine as well as carbachol. Cytosolic calcium concentrations begin to rise 50 min later and go off-scale at 66 min with over a 30-fold increase.

FIG. 14 shows a series of images obtained during Fura-2 calcium imaging studies with serum-starved ASM stimulated with 12 μg/ml pIgA. The cell that is circled in Frame 1 contracts and disappears by Frame 11.

FIG. 15 shows a western blot confirming that the different scFv clones recognize purified J-chain protein.

FIG. 16A is a graph showing that pIgA causes a dose-dependent rise in cytosolic calcium in Fura2-loaded serum-starved human ASM grown in 96-well fluorescence plates.

FIG. 16B shows that a mouse monoclonal anti-J-chain antibody decreases pIgA-induced increases in cystolic calcium concentration in Fura2-loaded serum-starved human ASM grown in 96-well fluorescence plates.

FIG. 17 is a graph showing that pIgA increases tension in isolated strips of dog tracheal smooth muscle as recorded at 5.5 h.

FIG. 18 is a graph showing increased tension responses to histamine after incubating dog tracheal smooth muscle strips with pIgA for 4 h.

### DETAILED DESCRIPTION OF THE INVENTION

I. Therapeutic Methods

As mentioned above, the present inventors have determined that IgA receptors, such as Fc-alpha R, are present on RA and OA synovial fibroblasts as well as synovial tissue from arthritis patients and that binding the receptor stimulates inflammatory mediator production in synovial fibroblast cells. Non-diseased human skin fibroblasts and pulmonary fibroblasts do not express IgA receptors.

The present inventors have also determined that IgA receptors, including pIgR and Fc-alpha R, are present on ASM cells and that binding the receptor causes calcium signalling. Therefore the present invention includes all diagnostic and therapeutic methods for treating conditions that are mediated through modulation of signalling through IgA receptors on mesenchymal cells.

Broadly stated, the present invention provides a method of modulating the inflammatory responses of a mesenchymal cell comprising administering to a cell or animal in need thereof an effective amount of an agent that can modulate an IgA receptor on a mesenchymal cell.

The present invention also includes a use of an effective amount of an agent that can modulate an IgA receptor on a mesenchymal cell to modulate the inflammatory responses of a mesenchymal cell. The invention further includes a use of an effective amount of an agent that can modulate an IgA receptor on a mesenchymal cell for the manufacture of a medicament to modulate the inflammatory responses of a mesenchymal cell.

The term “modulate” as used herein includes the inhibition or suppression of a function or activity as well as the induction or enhancement of a function or activity and interference with the interaction between any isoform of IgA and its receptor such as pIgR or FcαR. For example, an agent that can modulate IgA receptors includes agents that can inhibit or block the signalling through this receptor (receptor antagonists) as well as agents that can induce or stimulate signalling through the receptor (receptor agonists).

The term “IgA receptor” means any receptor on a mesenchymal cell that can bind an isoform of IgA. The receptor may also bind other immunoglobulins. In a preferred embodiment, the IgA receptor on the mesenchymal cell is pIgR or FcαR.

The term “pIgR” as used herein denotes a polymeric immunoglobulin receptor and means a receptor on cells that binds polymeric IgA (pIgA), dimeric IgA (dIgA) and polymeric IgM (pIgM) but not monomeric forms of IgA. The term includes the pIgR that has been previously described on epithelial cells (Piskurich et al., J. Immuno. 154:1735-1747, 1995) as well as any analogs, homologues, derivatives or variants (including splice variants) of the known pIgR molecules.

The term “FcαR” as used herein denotes the Fc-alpha receptor, also known as CD89, and means a receptor on cells that binds any isoform of IgA by its Fc portion. The term includes the FcαR that has been previously described on white blood cells (Morton et al, Crit. Rev. Immunol. 16: 423-440, 1996) as well as any analogs, homologues, derivatives or variants (including splice variants) of the known FcαR molecules.

The term “mesenchymal cell” as used herein includes fibroblasts, synovial cells, smooth muscle cells and endothelial cells. The mesenchymal cell will express pIgR or a pIgR-like protein and/or the Fc-alpha receptor. The mesenchymal cell is preferably a synovial fibroblast cell or an airway smooth muscle cell.

The term “a cell” as used herein includes a single cell as well as a plurality or population of cells. Administering an agent to a cell includes both in vitro and in vivo administrations.

The term “animal” as used herein includes all members of the animal kingdom, including humans. Preferably, the animal to be treated is a human.

The term “effective amount” as used herein means an amount effective, at dosages and for periods of time necessary to achieve the desired result, e.g. to modulate cell inflammatory responses or to modulate calcium signalling.

The IgA receptor antagonist (such as a pIgR or FcαR antagonist) can be any agent that inhibits signalling through the IgA receptor and results in an inhibition of function caused by signalling through the receptor including an inhibition of cellular inflammation or an inhibition of pIgR- or FcαR-mediated endocytosis. In one embodiment, the IgA receptor antagonist will inhibit the binding of pIgA to pIgR or FcαR on mesenchymal cells. The IgA receptor antagonist may be an antibody that binds, but does not activate the pIgR or FcαR on mesenchymal cells, and results in an inhibition of the binding of IgA with the resultant inhibition of cellular inflammation or cytosolic calcium signalling. Other IgA receptor antagonists include anti-J chain antibodies that might interfere with the ability of pIgR to bind pIgA or pIgM or antibodies or ligands to the portion of the Fc-alpha part of IgA that binds to Fc-alpha receptor on airway smooth muscle cells. Examples of other pIgR or FcαR antagonists are provided in Section II.

In one embodiment, the present invention provides a method of preventing or inhibiting the inflammatory responses of a mesenchymal cell comprising administering an effective amount of an IgA receptor antagonist to a cell or animal in need thereof. The present invention also provides a use of an effective amount of an IgA receptor antagonist to prevent or inhibit the inflammatory responses of the mesenchymal cell. The invention further includes a use of an effective amount of an IgA receptor antagonist in the manufacture of a medicament to prevent or inhibit the inflammatory responses of a mesenchymal cell.

The term “preventing or inhibiting the inflammatory responses of a mesenchymal cell” means that the inflammatory responses of the mesenchymal cell in the presence of the IgA receptor antagonist is decreased as compared to the level of inflammatory response in the absence of the antagonist. Inflammatory responses of mesenchymal cells can be measured using a variety of techniques known in the art including the techniques as described in the examples.

The methods of the invention can be used to treat any condition wherein it is desirable to modulate IgA receptor activity on mesenchymal cells. Such conditions include, but are not limited to, inflammatory diseases including arthritides (including rheumatoid arthritis, osteoarthritis, spondyloarthropathies), asthma, Crohn's disease, ulcerative colitis, Behcet's disease, Sjogren's disease and vasculitides.

Accordingly, the present invention provides a method of treating an inflammatory condition caused by IgA binding to an IgA receptor on a mesenchymal cell comprising administering an effective amount of an IgA receptor antagonist to a patient in need thereof. The present also includes the use of an effective amount of an IgA receptor antagonist to treat an inflammatory condition. The invention further includes a use of an effective amount of an IgA receptor antagonist in the manufacture of a medicament to treat an inflammatory condition.

As used herein, and as well understood in the art, “treating” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging survival as compared to expected survival if not receiving treatment.

The inventors have shown that pIgA stimulates NF-κB activity in RA synovial fibroblasts. As RA is characterized by increased NF-κB activity inhibiting the activity of this pro-inflammatory transcription factor (by inhibiting an IgA receptor) may be useful in treating arthritis. The inventors have also shown that IgA increases TNF-α induced ICAM-1 expression on synovial fibroblast and also increases TNF-α induced gene expression in rheumatoid arthritis and osteoarthritis synovial fibroblasts. The inventors have also shown that immune complexes of IgA and IgG increase ICAM-1 expression in synovial fibroblasts. Accordingly, the present invention provides a method of treating a patient with arthritis comprising administering an effective amount of an IgA receptor antagonist to a patient in need thereof. The invention further provides the use of an effective amount of an IgA receptor antagonist to treat a patient with arthritis. The present invention further includes a use of an effective amount of an IgA receptor antagonist in the manufacture of a medicament to treat a patient with arthritis.

As mentioned previously, the binding of IgA to an IgA receptor induces intracellular calcium signalling which further induces a variety of calcium dependent effects. Accordingly, the present invention provides a method of preventing or inhibiting intracellular calcium signalling in a mesenchymal cell comprising administering an effective amount of an IgA receptor antagonist to a cell or animal in need thereof. The invention further includes a use of an effective amount of an IgA receptor antagonist to prevent or inhibit intracellular calcium signalling in a mesenchymal cell. The invention further includes a use of an effective amount of an IgA receptor antagonist in the manufacture of a medicament to prevent or inhibit intracellular calcium signalling in a mesenchymal cell.

The term “preventing or inhibiting intracellular calcium signalling” means that the intracellular level of calcium in a mesenchymal cell in the presence of the a IgA receptor antagonist is decreased as compared to the level of intracellular calcium in cells in the absence of the agent. Calcium levels can be measured using a variety of known techniques including using fluorescence spectrophotometric and imaging techniques.

Intracellular calcium signalling is important for several processes in cell biology, including cell division, cytokine/chemokine/growth factor production, cell movement and contraction. Therefore, inhibiting calcium signalling can inhibit a variety of calcium dependent effects. Accordingly, the present invention provides a method of inhibiting the contraction of a mesenchymal cell comprising administering an effective amount of an IgA receptor antagonist to a cell or animal in need thereof.

The methods of the invention can be used to treat any condition wherein it is desirable to modulate IgA receptor (such as pIgR or FcαR) activity in order to prevent calcium signalling and thereby inhibit a variety of calcium dependent effects in airway smooth muscle cells. Such conditions include, but are not limited to asthma, chronic bronchitis, acute bronchitis, bronchial hyperreactivity, chronic obstructive pulmonary disease, emphysema, interstitial lung disease, bronchiectasis or airway remodelling.

Accordingly, the present invention provides a method of treating a condition wherein it is desirable to inhibit a calcium dependent effect in an airway smooth muscle cell comprising administering an effective amount of an IgA receptor antagonist to an animal in need thereof. The invention also includes a use of an effective amount of an IgA receptor antagonist to treat a condition wherein it is desirable to inhibit a calcium dependent effect in an airway smooth muscle cell. The present invention also includes a use of an effective amount of an IgA receptor antagonist for the manufacture of a medicament to treat a condition wherein it is desirable to inhibit a calcium dependent effect in an airway smooth muscle cell.

In a preferred embodiment, the method is useful in treating asthma. It is possible that IgA-induced calcium signalling may directly influence smooth muscle contraction and thereby contribute to bronchial hyperreactivity in asthma. It is also possible that IgA causes activation of transcription factors (e.g. NFκB) important for inflammatory reactions and subsequent production of cytokines, chemokines, adhesion molecules and growth factors. As a result, inhibition of IgA-induced calcium signalling may greatly improve the degree of inflammation in asthma as well as the airway remodelling described in chronic asthmatics. Furthermore, it is possible that this IgA-related phenomenon may account, at least in part, for the wide variety of clinical presentations and therapeutic responsiveness in patients with asthma.

Accordingly, the present invention provides a method of treating a patient with asthma comprising administering an effective amount of an IgA receptor antagonist to a patient in need thereof. The invention further provides the use of an effective amount of an IgA receptor antagonist to treat a patient with asthma. The present invention further includes a use of an effective amount of an IgA receptor antagonist in the manufacture of a medicament to treat a patient with asthma.

The present invention further provides a method of inhibiting the production of inflammatory mediators or growth factors comprising. administering an effective amount of an IgA receptor antagonist to a cell or animal in need thereof. In a preferred embodiment, the method inhibits the production of NF-κB. The present invention provides a use of an effective amount of an IgA receptor antagonist to inhibit the production of inflammatory mediators or growth factors. The present invention further provides a use of an effective amount of an IgA receptor antagonist in the manufacture of a medicament to inhibit the production of inflammatory mediators or growth factors.

II. Agents that Modulate pIgR or FcαR

The finding by the present inventors that pIgR or FcαR are on mesenchymal cells allows the discovery and development of agents that modulate pIgR or FcαR for use in modulating diseases mediated through an IgA receptor, such as pIgR or FcαR, on mesenchymal cells.

The present invention includes the use of any and all agents that modulate pIgR or FcαR in the methods of the invention. The agent can be any type of substance, including, but not limited to, nucleic acids (including antisense oligonucleotides, proteins (including antibodies), peptides, peptide mimetics, carbohydrates, organic compounds, inorganic compounds, small molecules, drugs, pIgR or FcαR ligands, soluble forms of pIgR or FcαR, pIgR or FcαR agonists, pIgR or FcαR antagonists, agents that inhibit pIgR or FcαR agonists, polymeric IgA (pIgA), dimeric IgA (dIgA) and polymeric IgM (pIgM) and fragments of these IgA or IgM molecules. Examples of some of the agents that modulate pIgR or FcαR are provided below.

(I) Antibodies

In one embodiment, the agent that can modulate pIgR is an antibody that binds to pIgR. Within the context of the present invention, antibodies are understood to include monoclonal antibodies, polyclonal antibodies, antibody fragments (e.g., Fab, Fab′, F(ab′)₂, scFv and Fv fragments) and recombinantly produced binding partners. Antibodies to pIgR may act as pIgR agonists or pIgR antagonists. For example, whole antibodies may act as pIgR agonists by stimulating the receptor while antibody fragments may act as pIgR antagonists by blocking the ability of pIgR ligands (such as pIgA) to bind pIgR.

In one embodiment, the antibody is an antibody fragment that acts as a pIgR antagonist. In a specific embodiment, the antibody fragment is a single chain Fv antibody. Single chain-Fv fragments can be isolated from a phage display library. The preparation of scFv antibodies to pIgR is described in Example 2. In Example 4, the inventors demonstrate that a mouse monoclonal IgG1 anti-J chain antibody decreases the pIgA induced increase in cystolic calcium in airway smooth muscle cells.

In one embodiment, the agent that can modulate FcαR is an antibody that binds to FcαR. Within the context of the present invention, antibodies are understood to include monoclonal antibodies, polyclonal antibodies, antibody fragments (e.g., Fab, Fab′, F(ab′)₂, scFv and Fv fragments) and recombinantly produced binding partners. Antibodies to FcαR may act as FcαR agonists or FcαR antagonists. For example, whole antibodies may act as FcαR agonists by stimulating the receptor while antibody fragments may act as FcαR antagonists by blocking the ability of FcαR ligands (such as mIgA or pIgA) to bind FcαR. Whereas several IgG, mouse monoclonal antibodies (A3, A59, A62 and A77) recognize the FcαR, only the mouse IgM monoclonal antibody My43 blocks the IgA binding site on the receptor (Kerr and Woof, 1999). My43 which cross-links the FcαR has also been shown to trigger a respiratory burst in neutrophils and monocytes as well as the release of calcium from intracellular stores in neutrophils (MacKenzie and Kerr, 1995; Shen, 1992; Stewart et al, 1994).

In one embodiment, the antibody is an antibody fragment that acts as a FcαR antagonist. In a specific embodiment, the antibody fragment is a single chain Fv antibody. Single chain-Fv fragments can be isolated from a phage display library. The preparation of scFv antibodies to FcαR is described in Example 2.

In another embodiment, the antibody is a pIgR agonist. Examples of antibodies that are pIgR agonists include pIgA and pIgM. Antibodies to mesenchymal pIgR may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

In another embodiment, the antibody is a FcαR agonist. Examples of antibodies that are FcαR agonists include mIgA and pIgA. Antibodies to mesenchymal FcαR may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

(ii) Antisense Oligonucleotides

In another embodiment, the agent that can modulate pIgR or FcαR is an antisense oligonucleotide that acts as a pIgR or FcαR antagonist, respectively, by inhibiting the expression of the pIgR or FcαR gene. The term “antisense oligonucleotide” as used herein means a nucleotide sequence that is complimentary to its target, e.g. the pIgR or FcαR gene. The sequence of the pIgR and FcαR genes are known in the art for many species, for example, see Piskurich et al., J. Immunol. 154:1735-1747, 1995, and Maliszewski et al, J. Exp. Med. 172:1665-1672, 1990.

The term “oligonucleotide” as used herein refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine. 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

(iii) Peptide Mimetics

The present invention also includes peptide mimetics of the pIgR or FcαR proteins. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a pIgR peptide, or enhancer or inhibitor of the pIgR peptide. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

(iv) Other Substances

In addition to antibodies and antisense oligonucleotides, other substances that can modulate pIgR or FcαR can also be identified and used in the methods of the invention. In one embodiment, the pIgR or FcαR modulator is a protein or peptide that can bind to pIgR or FcαR. The pIgR- or FcαR-binding peptides may be isolated by assaying a sample for peptides that bind to pIgR or FcαR. Any assay system or testing method that detects protein-protein interactions may be used including co-immunoprecipitation, crosslinking and co-purification through gradients or chromatographic columns may be used. Biological samples and commercially available libraries may be tested for pIgR- or FcαR-binding peptides. For example, labelled pIgR or FcαR may be used to probe phage display libraries. In addition, antibodies that bind pIgR or FcαR may be used to isolate other peptides with pIgR or FcαR binding affinity. For example, labelled antibodies may be used to probe phage display libraries or biological samples. Additionally, a DNA sequence encoding a pIgR protein may be used to probe biological samples or libraries for nucleic acids that encode pIgR- or FcαR-binding proteins.

Substances which can bind pIgR or FcαR may be identified by reacting pIgR or FcαR, respectively, with a substance which potentially binds to pIgR or FcαR, then detecting if complexes between the respective receptor and the substance have formed. Substances that bind pIgR or FcαR in this assay can be further assessed to determine if they are useful in modulating or inhibiting pIgR or FcαR and useful in the therapeutic methods of the invention.

Accordingly, the present invention also includes a method of identifying substances which can bind to mesenchymal pIgR or FcαR comprising the steps of:

(a) reacting pIgR or FcαR on a mesenchymal cell and a test substance, under conditions which allow for formation of a complex between the pIgR or FcαR and the test substance, and

(b) assaying for complexes of pIgR or FcαR and the test substance, for free substance or for non complexed pIgR or FcαR, wherein the presence of complexes indicates that the test substance is capable of binding pIgR or FcαR.

Conditions which permit the formation of substance and IgA receptor complexes may be selected having regard to factors such as the nature and amounts of the substance and the protein.

The substance-IgA receptor complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against pIgR or FcαR or the substance, or labelled pIgR or FcαR, or a labelled substance may be utilized. The antibodies, pIgR or FcαR, or substances may be labelled with a detectable substance.

The pIgR or FcαR gene or protein may be used as a target for identifying lead compounds for drug developments. The invention therefore includes an assay system for determining the effect of a test compound or candidate drug on the activity of the pIgR or FcαR gene or protein.

Accordingly, the present invention provides a method for identifying a compound that modulates mesenchymal pIgR or FcαR activity comprising:

(a) incubating a test compound with a mesenchymal pIgR or FcαR protein or a nucleic acid encoding a mesenchymal pIgR or FcαR protein; and

(b) determining the effect of the test compound on the pIgR or FcαR protein activity or pIgR or FcαR gene expression and comparing with a control (i.e. in the absence of a test compound) wherein a change in the pIgR or FcαR protein activity or pIgF3 or FcαR gene expression as compared to the control indicates that the test compound is a potential modulator of the pIgR or FcαR gene or protein.

In one embodiment, pIgR or FcαR activity may be assessed by measuring intracellular calcium levels as previously described.

III. Compositions

The present invention also includes pharmaceutical compositions containing the agents that can modulate or inhibit pIgR or FcαR for use in the methods of the invention. Accordingly, the present invention provides a pharmaceutical composition for modulating the inflammatory responses of a mesenchymal cell comprising an effective amount of an agent that can modulate an IgA receptor in admixture with a suitable diluent or carrier. The present invention also includes a pharmaceutical composition for preventing or inhibiting the inflammatory responses of a mesenchymal cell comprising an effective amount of an IgA receptor antagonist in admixture with a suitable diluent or carrier. The present invention further provides a pharmaceutical composition for preventing or treating arthritis comprising an effective amount of an IgA receptor antagonist in admixture with a suitable diluent or carrier. In a preferred embodiment, the IgA receptor antagonist is a pIgR or FcαR antagonist.

Such pharmaceutical compositions can be for intralesional, intravenous, topical, rectal, parenteral, local, inhalant or subcutaneous, intradermal, intramuscular, intra-articular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other agents such as other agents that can modulate or inhibit cell inflammatory responses or that are used in treating inflammatory conditions such as arthritis and asthma.

IV. Targeted Delivery

The finding by the present invention that pIgR and FcαR are on mesenchymal cells allows the development of methods to target the delivery of substances directly to mesenchymal cells. Accordingly, the present invention provides a method of delivering a substance to a mesenchymal cell comprising administering an effective amount of a conjugate comprising the substance coupled to an IgA receptor ligand to an animal or cell in need thereof.

The substance can be any substance that one wishes to deliver, including therapeutics and diagnostics to a mesenchymal cell in a specific embodiment, the substance is useful in treating an inflammatory condition such as arthritis.

The ligand can be any molecule that can bind the IgA receptor, including pIgA or pIgM, as well as the ligands described in Section II.

The substance may be coupled to the IgA receptor ligand either directly or indirectly. In direct coupling, the substance and ligand are physically linked such as by covalent binding or physical forces such as van der Waals or hydrophobic interactions. In indirect coupling, the substance and ligand are joined through another molecule or linker. As one example, the substance and ligand may be joined through a bispecific antibody that binds both the substance and linker.

Conjugates of the substance arid the IgA receptor ligand may be prepared using techniques known in the art. There are numerous approaches for the conjugation or chemical crosslinking of proteins and one skilled in the art can determine which method is appropriate for the substance to be conjugated. The method employed must be capable of joining the substance with the IgA receptor ligand without interfering with the ability of the ligand to bind to the IgA receptor and without significantly altering the activity of the substance. If the substance and ligand are both proteins, there are several hundred crosslinkers available in order to conjugate the substance with the ligand. (See for example “Chemistry of Protein Conjugation and Crosslinking”. 1991, Shans Wong, CRC Press, Ann Arbor). The crosslinker is generally chosen based on the reactive functional groups available or inserted on the substance. In addition, if there are no reactive groups a photoactivatible crosslinker can be used. In certain instances, it may be desirable to include a spacer between the substance and the ligand. In one example, the ligand and substance may be conjugated by the introduction of a sulfhydryl group on the ligand and the introduction of a cross-linker containing a reactive thiol group on to the substance through carboxyl groups (Wawizynczak, E. J. and Thorpe, P. E. in Immunoconjugates: Antibody Conjugates in Radioimaging and Therapy of Cancer, C. W. Vogel (Ed.) Oxford University Press, 1987, pp. 28-55.; and Blair, A. H. and T. I. Ghose, J. Immunol. Methods 59:129, 1983).

In another embodiment, the protein ligand and substance may be prepared as a fusion protein. Fusion proteins may be prepared using techniques known in the art. In such a case, a DNA molecule encoding the IgA receptor ligand is linked to a DNA molecule encoding the substance. The chimeric DNA construct, along with suitable regulatory elements can be cloned into an expression vector and expressed in a suitable host.

The conjugates of the invention may be tested for their ability to enter mesenchymal cells and provide the desired pharmacological effect using in vitro and in vivo models.

V. Diagnostic Assays

The finding by the present inventors that airway smooth muscle cells have IgA receptors (such as pIgR and FcαR) allows the development of diagnostic assays to detect IgA mediated diseases. Such diagnostic assays can facilitate the development of tailored therapies for such diseases. In one example, patients can undergo bronchial challenge testing to determine the presence of IgA-mediated bronchial hyperreactivity. Methacholine challenge and histamine challenge testing are examples of currently used tests to evaluate respiratory symptoms of cough, wheeze and shortness of breath, and to detect nonspecific bronchial hyperresponsiveness as demonstrated by exaggerated bronchoconstriction to inhaled methacholine or histamine. Pre- and post-challenge pulmonary function test measurements of forced expiratory volume at 1 second (FEV1) and forced vital capacity (FVC) could be done every 5-10 minutes following bronchial challenge with an agonist to IgA receptors that would result in increased intracellular calcium concentrations in ASM and consequent bronchoconstriction. The IgA receptor agonist can be any substance that will increase airway smooth muscle cytosolic calcium concentrations and contraction which would manifest clinically as bronchoconstriction. This bronchoconstriction can be detected subjectively by hearing wheezing on chest auscultation, or objectively by measuring a reduced FEV1 by standard spirometry or reduced peak flow. For example, bronchoconstriction may be detected by a drop in FEV1. IgA-mediated bronchial hyperreactivity could be graded according to the concentration of the test substance that results in a 20% fall in baseline FEV1. Accordingly, the present invention provides a method of detecting IgA mediated bronchial hyperreactivity comprising:

(a) administering an IgA receptor agonist to a patient; and

(b) detecting bronchoconstriction in the patient wherein an increase in bronchoconstriction as compared to a control indicates that the patient has IgA-mediated bronchial hyperreactivity.

Another example of a diagnostic test would be to perform a nonspecific bronchial challenge test with either methacholine or histamine on one day, and then repeat this challenge on another day after pretreating patients with an IgA receptor agonist. IgA-mediated bronchial hyperreactivity would be detected by a significant increase sensitivity to the non-specific bronchoconstrictor (i.e. a lower dose of methacholine or histamine induces the 20% fall in FEV1 from baseline measurement). Another example would be to perform a bronchial challenge test with both a nonspecific bronchoconstrictor and an IgA receptor agonist.

Accordingly, the present invention provides a method of detecting IgA-mediated bronchial hyperreactivity comprising:

(a) administering an IgA-receptor agonist to a patient and detecting bronchoconstriction; and

(b) administering an IgA receptor agonist followed by a non-specific bronchoconstricting agent to the patient at a lower dose than when the nonspecific agent is administered alone and detecting bronchoconstriction wherein bronchoconstriction in step (a) and/or bronchoconstriction induced at a lower dose of the nonspecific agent administered without the IgA receptor agonist in step (b) would indicate that the patient has IgA-mediated bronchial hyperreactivity.

In a bronchial challenge test, a patient inhales increasing amounts of the agent. The patient's FEV1 and FVC are measured by spirometry 30-90 seconds after inhaling the agent. If there is no significant (=20% change from baseline), the next dose is administered 5 minutes later, but no sooner than 5 minutes. The challenge is stopped when the patient develops a 20% drop in baseline FEV1 (—which indicates bronchoconstriction—or FVC) or a specific maximum dose has been given (=8 mg/ml in the case of methacholine). (Higher doses of methacholine will cause bronchoconstriction in nearly everyone. People who react to doses less than 8 mg/ml are categorized as having severe, moderate, mild or borderline hyperreactivity to the agent depending on the dose of the agent that causes the 20% drop in FEV1. These challenge tests are used to diagnose bronchial hyperreactivity and monitor therapy. Note that narrowing might occur in very distal airways without narrowing in more proximal airways and that this very distal narrowing might not result in changes in FEV1 but would be suggested by a decrease in FEF_{25-75%} (forced expiratory flow during 25 to 75% of the forced vital capacity).

The finding by the present inventors that synovial fibroblasts and endothelial cells in synovial tissue from arthritis patients as well as airway smooth muscle cells have IgA receptors (such as pIgR and FcαR) allows the development of diagnostic assays to detect diseases mediated through IgA binding to an IgA receptor on a mesenchymal cell. Such diagnostic assays can facilitate the development of tailored therapies for such diseases. Samples from patients can be obtained and tested for the presence of IgA receptors, such as pIgR or FcαR, on mesenchymal cells. The sample can be any sample that contains a mesenchymal cell including synovial fibroblasts and synovial tissue, connective tissue, endothelial cells and blood vessels, smooth muscle cells, or primary cell cultures of these cells derived from a tissue biopsy. Patients expressing an IgA receptor may be treated with IgA receptor antagonists as described above.

Accordingly, the present invention provides a method of detecting a condition associated with the activation of an IgA receptor on a mesenchymal cell comprising assaying a sample for (a) a nucleic acid molecule encoding an IgA receptor or a fragment thereof or (b) an IgA receptor or a fragment thereof. The IgA receptor is preferably pIgR or FcαR. In one embodiment, the condition associated with the activation of an IgA receptor on a mesenchymal cell is an Inflammatory condition such as arthritides (including rheumatoid arthritis, osteoarthritis, spondyloarthropathies), asthma, Crohn's disease, ulcerative colitis, Behcet's disease, Sjogren's disease and vasculitides.

(i) Detecting Nucleic Acid Molecules Encoding IgA Receptors

Nucleotide probes can be prepared and used in the detection of nucleotide sequences encoding an IgA receptor or fragments thereof in samples, preferably pIgR or FcαR. The probes can be useful in detecting the presence of a condition associated with the activation of an IgA receptor on a mesenchymal cell or monitoring the progress of such conditions include inflammatory conditions including the arthritides (including rheumatoid arthritis, osteoarthritis, spondyloarthropathies), Crohn's disease, ulcerative colitis, Behcet's disease and Sjogren's disease and vasculitides. Accordingly, the present invention provides a method for detecting a nucleic acid molecule encoding an IgA receptor comprising contacting the sample with a nucleotide probe capable of hybridizing with the nucleic acid molecule to form a hybridization product, under conditions which permit the formation of the hybridization product, and assaying for the hybridization product.

The nucleotide probe may be labelled with a detectable substance such as a radioactive label which provides for an adequate signal and has sufficient half-life such as 32P, 3H, 14C or the like. Other detectable substances which may be used include antigens that are recognized by a specific labelled antibody, fluorescent compounds, enzymes, antibodies specific for a labelled antigen, and chemiluminescence. An appropriate label may be selected having regard to the rate of hybridization and binding of the probe to the nucleic acid to be detected and the amount of nucleic acid available for hybridization. Labelled probes may be hybridized to nucleic acids on solid supports such as nitrocellulose filters or nylon membranes as generally described in Sambrook et al, 1989, Molecular Cloning, A Laboratory Manual (2nd ed.).

Nucleic acid molecules encoding an IgA receptor can be selectively amplified in a sample using the polymerase chain reaction (PCR) methods and cDNA or genomic DNA. A nucleic acid can be amplified from cDNA or genomic DNA using oligonucleotide primers and standard PCR amplification techniques. The amplified nucleic acid can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

(ii) Detecting IgA Receptors

The presence of IgA receptors may be detected in a sample using IgA receptor ligands that bind to the IgA receptor. IgA receptor ligands are described above and include antibodies or other substances that can bind an IgA receptor. Accordingly, the present invention provides a method for detecting an IgA receptor comprising contacting the sample with a ligand that binds to an IgA receptor which is capable of being detected after it becomes bound to the IgA receptor in the sample.

Ligands to an IgA receptor, such as antibodies specifically reactive with an IgA receptor, or derivatives thereof, such as enzyme conjugates or labeled derivatives, may be used to detect an IgA receptor in various biological materials. For example they may be used in any known immunoassays which rely on the binding interaction between an IgA receptor, and an antibody thereof. Examples of such assays are radioimmunoassays, enzyme immunoassays (e.g. ELISA), immunofluorescence, immunoprecipitation, latex agglutination, hemagglutination and histochemical tests. Thus, the antibodies may be used to detect and quantify an IgA receptor in a sample in order to determine its role in particular cellular events or pathological states, and to diagnose and treat such pathological states, such as arthritis.

Cytochemical techniques known in the art for localizing antigens using light and electron microscopy may be used to detect an IgA receptor. Generally, an antibody of the invention may be labelled with a detectable substance and an IgA receptor may be localised in tissue based upon the presence of the detectable substance. Examples of detectable substances include various enzymes, fluorescent materials, luminescent materials and radioactive materials. Examples of suitable enzymes include horseradish peroxidase, biotin, alkaline phosphatase, β-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include radioactive iodine I-125, I-131 or 3-H. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against an IgA receptor. By way of example, if the antibody having specificity against an IgA receptor is a rabbit IgG antibody, the second antibody may be goat anti-rabbit gamma-globulin labelled with a detectable substance as described herein.

Where a radioactive label is used as a detectable substance, an IgA receptor may be localized by autoradiography. The results of autoradiography may be quantitated by determining the density of particles in the autoradiographs by various optical methods, or by counting the grains.

The following non-limiting examples are illustrative of the present invention:

### EXAMPLES

### Example 1

Primary Cell Cultures of RA and OA Synovial Fibroblasts Express FcαR

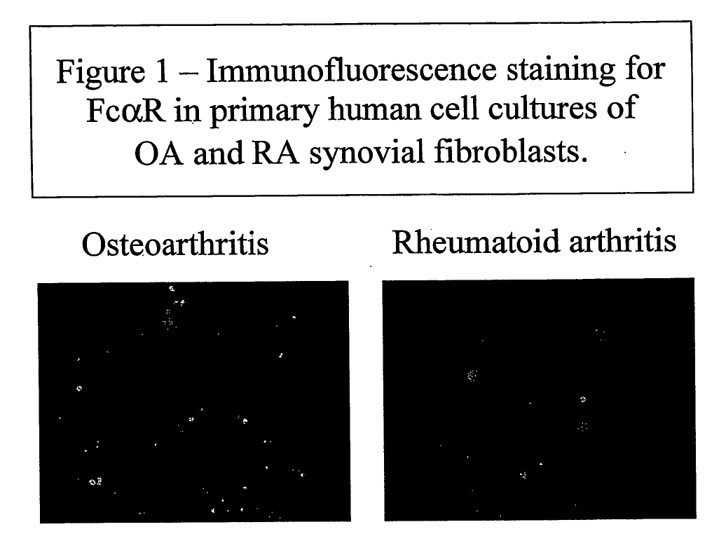

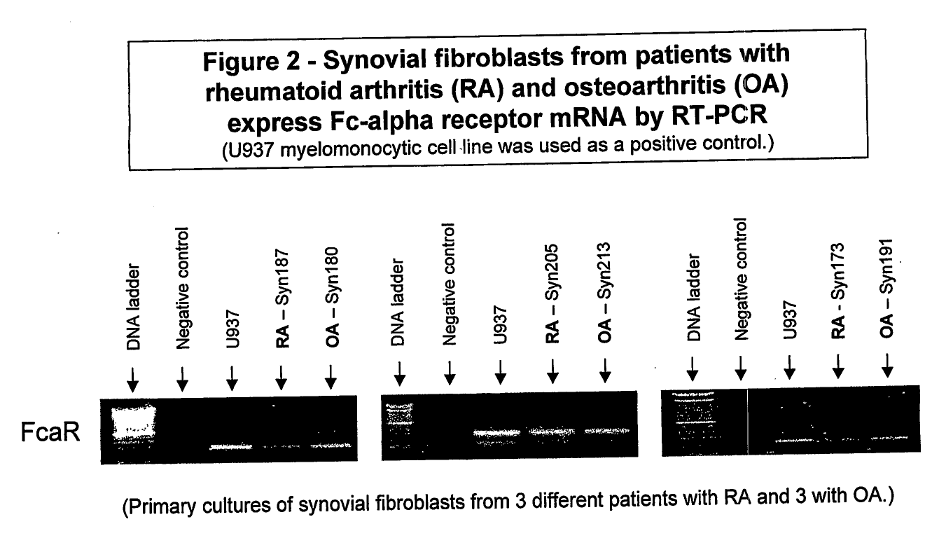

(a) FcαR protein expression was studied in primary cultures of RA and OA synovial fibroblasts by immunofluorescence. Tissues were obtained from patients undergoing joint replacement surgery and were digested with collagenase (1 mg/ml) and hyaluronidase (0.05 mg/ml) (Sigma) in Hanks' balanced salt solution (Gibco) for 1-2 h at 37 C. Cells were washed with 10% fetal bovine serum (FBS) in RPMI 1640 (Gibco), spun and grown in this same media for overnight culture at 37 C. Subsequently, cells were cultured in DMEM with 10% FBS, penicillin and streptomycin. Both RA and OA cells showed staining for FcαR using either a mouse (Santa Cruz) or goat (Santa Cruz) antibody to FcαR and the appropriate FITC-conjugated secondary antibody. FIG. 1 shows data obtained with the goat antibody to FcαR (Santa Cruz) (representative experiment of n=4 different subjects with RA and n=3 for OA).

Primary Cell Cultures of RA and OA Synovial Fibroblasts Express FcαR mRNA by RT-PCR

(b) FcαR mRNA expression was confirmed in both RA (n=9 different subjects) and OA (n=4) synovial fibroblasts by RT-PCR. FIG. 2 shows representative data from a total of 3 different patients with RA and 3 with OA. Primers for the the IgA binding domain of FcαR (sense: 5′ CCT CAG TCT GGG GCT TTC TTU 3′; antisense: 5′ CTT GTT TGC GTC CAT GTG GTC 3′) were used. The bands obtained from the RT-PCR product were DNA sequenced and confirmed to be sequences of their respective IgA receptors. These results confirm that RA and OA synovial fibroblasts express mRNA for FcαR.

Synovial Tissue from Patients with Arthritis Express Receptors for IgA.

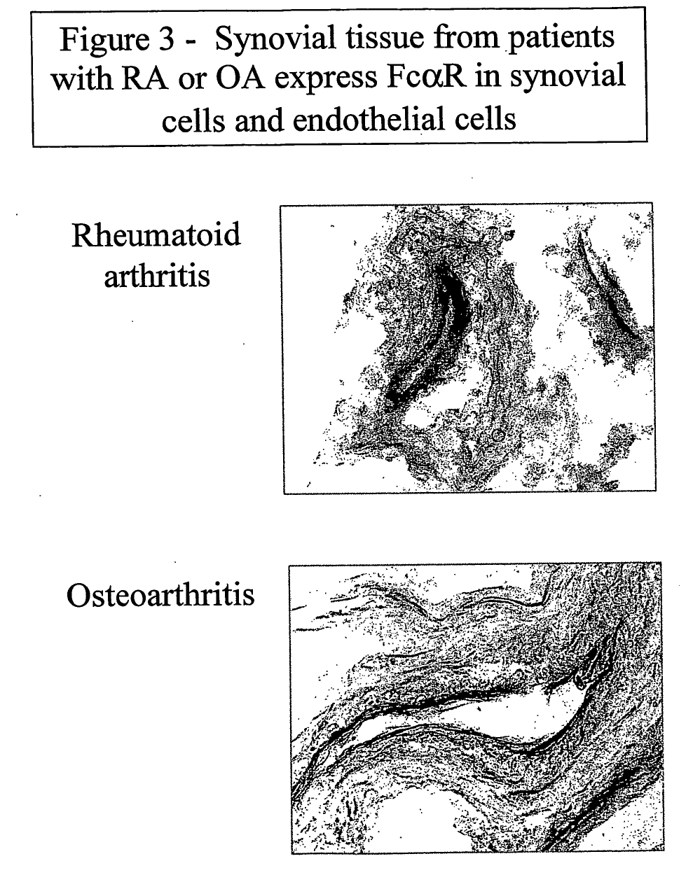

Acetone-fixed frozen sections of synovial tissue from arthritis patients undergoing joint replacement surgery were stained for IgA receptor expression. We have studied a total of 4 patients with RA and 3 other patients with OA using a goat polyclonal antibody to FcαR (Santa Cruz), and an HRP-conjugated secondary antibody (Jackson). FIG. 3 shows a representative slide confirming the presence of FcαR in synovial tissue from one patient each with RA or OA. Positive staining for FcαR can be seen in synovial cells within the tissue as well as on endothelial cells of blood vessels These results confirm that IgA receptor expression occurs in vivo in arthritis tissue as well as primary cell cultures of synovial fibroblasts. These results also show that a third type of mesenchymal cell—an endothelial cell—also expresses an IgA receptor,

IgA Stimulates NF-κB DNA Binding in RA and OA Synovial Fibroblasts.

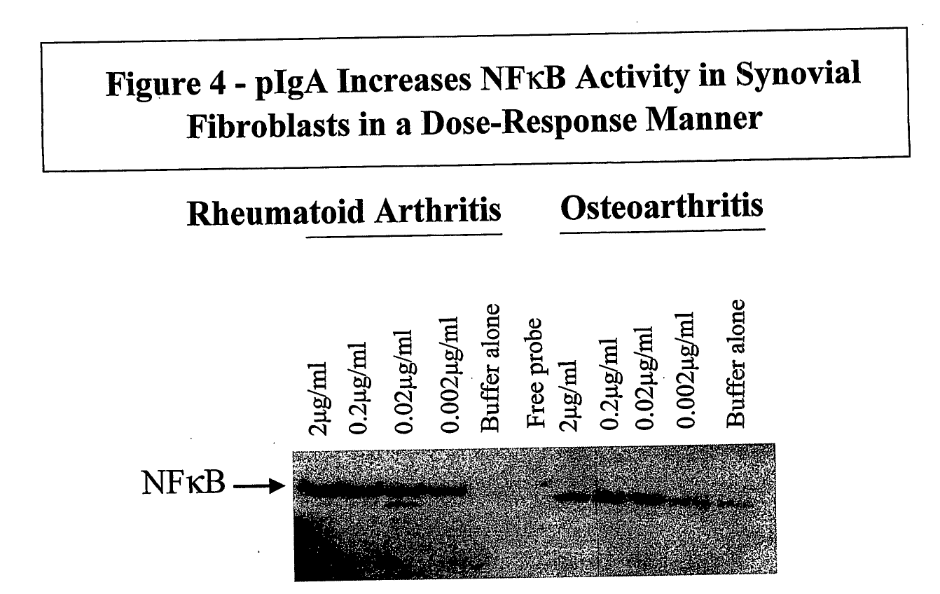

RA is characterized by increased activity of the pro-inflammatory transcription factor, NFκB, in synovial fibroblasts. Both RA and OA are chronic inflammatory conditions, but RA is an autoimmune inflammatory disease. To determine whether expression of IgA receptors might play a role in the inflammation of RA and OA, we asked whether pIgA stimulates NFκB DNA binding in RA and OA synovial fibroblasts. We found that pIgA induced a dose-dependent increase in NFκB activity in both RA and OA synovial fibroblasts by DNA electromobility gel shift assay (EMSA) (FIG. 4). EMSA was performed using the Promega gel shift assay system. The NFκB consensus oligonucleotide (5′]AGT TGA GGG GAC TTT CCC AGG C-3′) representing the p65 subunit was end-labeled with [γ-³²P]ATP using T4 polynucleotide kinase. EMSA was done using 5 μg of nuclear extract proteins and labeled oligonucleotide. The protein-DNA comples was separated on polyacrylamide gel, which was then exposed to autoradiographic film. This effect of IgA on increasing NFkB DNA binding in synovial fibroblasts has never been described and has major implications for the role of IgA receptors in RA and OA.

These results implicate synovial FcαR in the inflammatory processes of RA and OA.

### Example 2

scFv Selection Methods and Results:

A scFv phage library was reconstituted by pooling several first rounds of selection that the inventor had previously used. The scFv phage library that was originally used is described in: Sheets M D, Amersdorfer P, Finnern R, Sargent P, Lindquist E, Schier R, Hemingsen G, Wong C, Gerhart J C, Marks J D, Lindqvist E., Efficient construction of a large nonimmune phage antibody library: the production of high-affinity human single-chain antibodies to protein antigens. (Proc Natl Acad Sci USA. May 26, 1998;95(11):6157-62.).

TG-1/pHen/phage^{1st round} scFv. These selections were to domain of rat pIgR; and to cell selections for pIgR with MDCK cells transfected with rabbit pIgR and attempted in 12 different ways. These first round TG-1 from 13 different selections were combined and grown for isolating phage. These phage were used as the “reconstituted” phage library of scFv.

A. For Selections Against a Mesenchymal FcαR:

1. Coat 3 immunotubes with mIgA (Biolynx; 6.5 λ/3 ml PBS) and block with 2% milk.

2. Preclear reconstituted phage library twice with 2 of the coated immunotubes.

3. Incubate SDS lysates from non-serum-starved mesenchymal cells (e.g. airway smooth muscle cells, ASM, purchased from Biowhittaker) with the 3rd coated tube (—to bind the putative ASM FcαR to the mIgA).

4. Incubate precleared phage with the 3rd tube.

5. Wash extensively (15-20) with PBS and elute the bound phage with 1% TEA (triethanolamine). Neutralize the high pH with 1M Tris pH 7.4.

6. Infect TG-1 E. coli with the phage, and grow.

7. Expand and rescue phage to repeat procedure 2 more times.

8. After round 3, randomly pick 96 colonies and screen these for scFv production (dot blot) and cell ELISAs using (a) U937 cells (myelomonocytic cell line that highly expresses the FcαR); and (b) ASM, both cell lines grown in a 96-well plate.

Results:

8 positives by ASM ELISA (OD450>0.2); 3 positives by U937 cell ELISA
BstN1 DNA digest of pcr products from the 3 clones showed unique patterns, suggesting isolation of 3 different scFv
   B. For selections against a mesenchymal pIgR:

1. Coat 3 immunotubes with pIgA (10λ myeloma serum/3 ml PBS) and block with 2% milk PBS.

2. Preclear reconstituted phage library twice with 2 of the coated immunotubes.

3. Incubate SDS lysates from non-serum-starved mesenchymal cells (e.g. ASM, purchased from Biowhiftaker) with the 3rd coated tube (—to bind the putative ASM pIgR to the pIgA).

4. Incubate precleared phage with the 3rd tube.

5. Wash extensively (15-20) with PBS and elute the bound phage with 1% TEA. Neutralize with 1M Tris pH 7.4.

6. Infect TG-1 with the phage, and grow.

7. Expand and rescue phage to repeat procedure 2 more times.

8. After round 3, randomly pick 96 colonies and screen these for scFv production (dot blot) and cell ELISA using ASM and CALU-3 cells grown in a 96-well plate.

Results:

45 positives by ASM ELISA (includes 6 that were negative on CALU-3); 55 positives by CALU-3 ELISA.
Also screened by ELISA with hurrian milk which contains secretory component, the extracellular part of pIgR, and found 26 positives (used OD450>0.4 with background reading of ˜0.1); screened by ELISA with fetal calf serum-coated plate, and found 46 positives; rabbit anti-human SC antibody (Dako) used as positive control antibody
BstN1 DNA digest of pcr products from the all positive clones showed 12 unique patterns, suggesting isolation of 12 different scFv
   C. For Selections Against J-Chain:

Dr. Jiri Mestecky sent his PET32 plasmid containing the J-chain protein fused to thioredoxin and containing an IgA protease cleavage site and a 6His tag for purification (Symersky et al, 2000). This plasmid was infected into BL21 E. coli which were induced to produce the J-chain-thioredoxin fusion protein. This was purified by IMAC on a nickel resin.

1. Coat 2 immunotubes with thioredoxin (Sigma; 10 μg/ml) and block with 2% milk/PBS.

2. Coat 1 immunotube with the purified J-chain fusion protein, then block with 2% milk/PBS.

3. Preclear reconstituted phage library twice with the 2 thioredoxin-coated immunotubes.

4. Incubate precleared phage with the 3rd tube coated with the J-chain fusion protein.

5. Wash extensively (15-20) with PBS and elute the bound phage with TEA (triethanolamine). Neutralize the high pH with Tris buffer.

6. Infect [TG-1] E. coli with the phage, and grow.

7. Expand and rescue phage to repeat procedure 2 more times.

8. After round 3, randomly pick 96 colonies and screen these for scFv production (dot blot) and protein ELISA using one 96-well plate coated with thioredoxin and one plate coated with the J-chain fusion protein. (—To ensure that the scFv selected bind to J-chain and not to thioredoxin.)

Results:

30 positives by J-chain ELISA; none bound the thioredoxin-coated plate. (Background OD450 was ˜0.07; chose OD450>0.2 to be positive.)

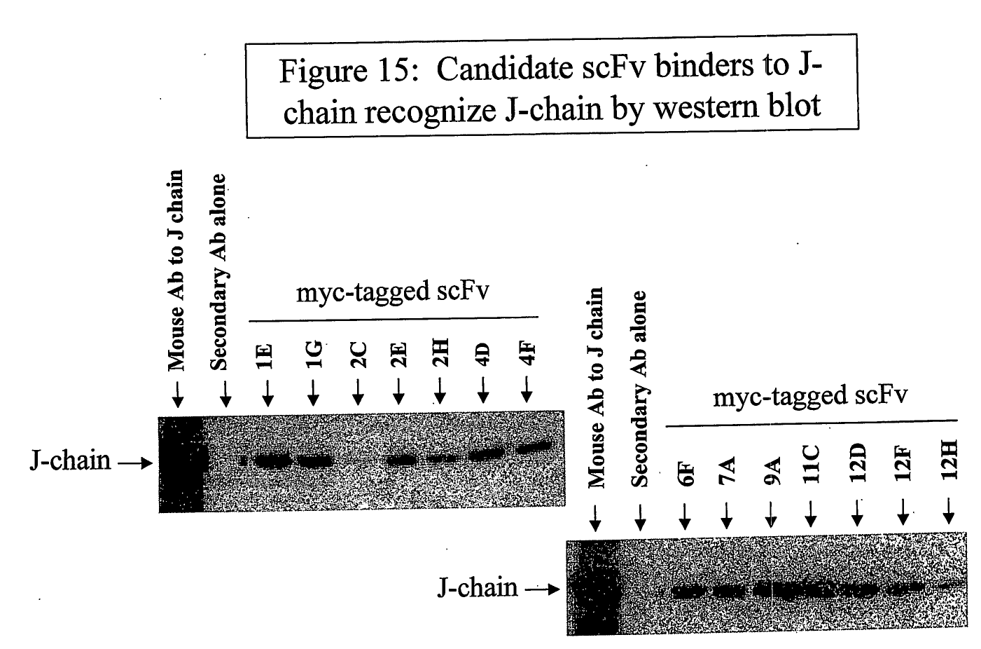

14 of these were induced to produce scFv (which contain a myc epitope tag) and all recognized J-chain by western blotting (mouse monoclonal anti-J-chain from InnoGenex was used as positive control); 9E10 (anti-myc mouse monoclonal antibody and anti-mouse HRP alone used as negative control). (FIG. 15)
BstN1 DNA digest of pcr products from all positive clones showed 5 unique patterns, suggesting isolation of 5 different scFv.
Of these 5 unique scFv binders to j-chain, 1 binds both protein L and protein A and 3 bind protein A. This characteristic constitutes additional evidence that these scFv differ from each other and provides another means to purify these scFv for further testing.

### Example 3

Methods

Cell Culture:

Primary human airway smooth muscle (ASM) cells from three different subjects were purchased from Clonetics (San Diego, Calif., USA) and grown in smooth muscle cell basal medium (SmBM) (Clonetics) supplemented with the smooth muscle cell SingleQuots (Clonetics), which containing 0.5 ng/ml human recombinant Epidermal Growth Factor (hEGF), 5 μg/ml insulin, 1 μg/ml human recombinant Fibroblast Growth Factor (hFGr), 50 μg/ml Gentamicin and 50 ng/ml Amphotericin-B, and 5% fetal calf serum. At 75% confluence, the cells were serum-deprived to induce differentiation (Halayko et al, 1999) and grown in serum-free basal media (Clonetics). Cells were studied between days 8 and 14 after serum starvation. ASM phenotype was confirmed by positive staining for markers of smooth muscle cell differentiation including myosin kinase light chain, and negative staining for factor VIII. ASM cells were used within the first 9 passages and perpetuated by trypsinizing cells for propagating at a 1:4 dilution.

Madin Darby canine kidney (MDCK) cells transfected with the cDNA for human pIgR were used as positive control cells (MDCK-HpIgR, from Dr. Charlotte Kaetzel) for detection of pIgR, and the myelomonocytic cell line, U937 (ATCC), was used for its high level of endogenous expression of Fc-alpha receptor.

Immunofluorescence Studies:

ASM are grown on collagen-coated coverslips in serum-containing growth medium to 70% conflurence and then replaced with serum-free medium for 8 to 14 days. After 16 hours treatment with pIgA, migA, rabbit anti-human secretory component (SC; Dako) or media alone, the coverslips were washed twice with ice cold PBS and fixed with 4% paraformaldehyde in PBS for 20 min on ice. After washed three times in PBS, the coverslips were blocked with 5% horse serum in PBS and 0.2% triton X-100 for 1 h at 37° C. Goat anti-alpha chain or rabbit anti-human SC at 1:100 dilution in blocking buffer were incubated with the coverslips for 1 h at 37° C. After washed three times in PBS and 0.05% triton X-100, the coverslips were incubated with secondary antibody FITC-labeled donkey anti-goat or donkey anti-rabbit at 1:200 dilution in blocking buffer for 45 min at 37° C. The coverslips then were mounted onto slides with one drop of mounting medium (Vector laboratories, Burlingame, Calif., USA) and fluorescence images were observed and captured with an Olympus fluorescence microscope with a digital camera. Live cell uptake of IgA was determined by staining fixed cells with a goat polyclonal antibody to the alpha chain of IgA 1:100 (Jackson Laboratories). FcαR staining was detected with a mouse monoclonal IgG1 antibody to the receptor 1:25 (BD Pharmingen) and a FITC-conjugated donkey anti-mouse IgG antibody.

Protein and RNA Expression:

Western blotting. ASM cells were lysed in 0.5% SDS lysis buffer with protease inhibitor (100 mM NaCl, 50 mM Tris [pH 8.1), 5 mM EDTA [pH 8.0], 0.02% NaN₃, 0.5% SDS, 1 mM PMSF, 10 μg/ml aprotinin, 1 mM Na₃VO₄) and run on a 8% SDS-PAGE gel. Lysates of MDCK-HpIgR were used as a positive control. After transferring to nitrocellulose membrane (Bio-Rad) for 1.5 h at 100 voltage in transfer buffer (25 mM Tris, 192 mM glycine, 20% methanol), Western blotting was performed using either a rabbit anti-human secretory component antibody (Dako) or a goat anti-human secretory component antibody (Sigma). The anti-SC antibody was detected with an HRP-labeled secondary goat antibody to rabbit IgG (Sigma) or donkey antibody to goat (Jackson Immunochemicals). Antibody binding was detected on film (Amersham Pharmacia Biotech) using enhanced chemiluminescence (ECL) (Amersham-Pharmacia Biotech).

RT-PCR and DNA sequencing. For detecting mRNA expression of pIgR or Fc-alpha receptor, total RNA was extracted from ASM, MDCK-HpIgR or U937 cells with TRIzol reagent (Life Technologies). Total RNA extracted from MDCK-HpIgR cells were used as a positive control for pIgR, while the U937 cells were used as the positive control for Fc-alpha receptor. Primers were designed to represent the cytoplasmic domain of pIgR and the IgA binding domain of the Fc-alpha receptor (Table 1 shows the sequence of the sense and antisense primers). RT-PCR was performed using oligo dT, mMLV reverse transcriptase and Taq DNA polymerase (Life Technologies). The RT-PCR products were run on a 1.2% agarose gel. Bands from the ASM lanes were cut out and purified with QIAquick Gel Extracton kit (Qiagen) for DNA sequencing. DNA sequences were compared to those in the Genebank of NCBI human genome database for identification.

Calcium Studies:

(1) Calcium measurements. ASM were grown in serum free media for 8 days on 96-well fluorescence plates (Costar). After loading cells with Fura 2-AM (Molecular Probe) for 3 h at 37° C., the ASM cells were treated in triplicate with either media alone, mIgA (12 μg/ml) or pIgA (12 μg/ml). Fluorescence was measured using a multiwell fluorescence plate reader (Molecular Dynamics) at excitation wavelengths of 340λ and 380λ and emission wavelengths at 500λ. Data was collected and calculated calcium concentration using the Grynkiewicz formula: [Ca²⁺]ᵢ (in nmol/L)=Kd [(R−Rₘᵢₙ)/(Rₘₐₓ−R)]×β factor. R is the ratio of fluorescence at 340 and 380 nm, Rₘₐₓ and Rₘᵢₙ are the ratios at 340 and 380 nm in the presence of saturating Ca²⁺ and zero Ca²⁺, β factor is the ratio at 380 nm in zero and saturating Ca²⁺, and Kd, the dissociation constant, is 224 nmol/L. At the end of the incubation, the conditions were washed off, and digitonin and EGTA were added to calculate the maximum and minimum cytosolic calcium concentrations, respectively. The results of this experiment are shown in Table 1.

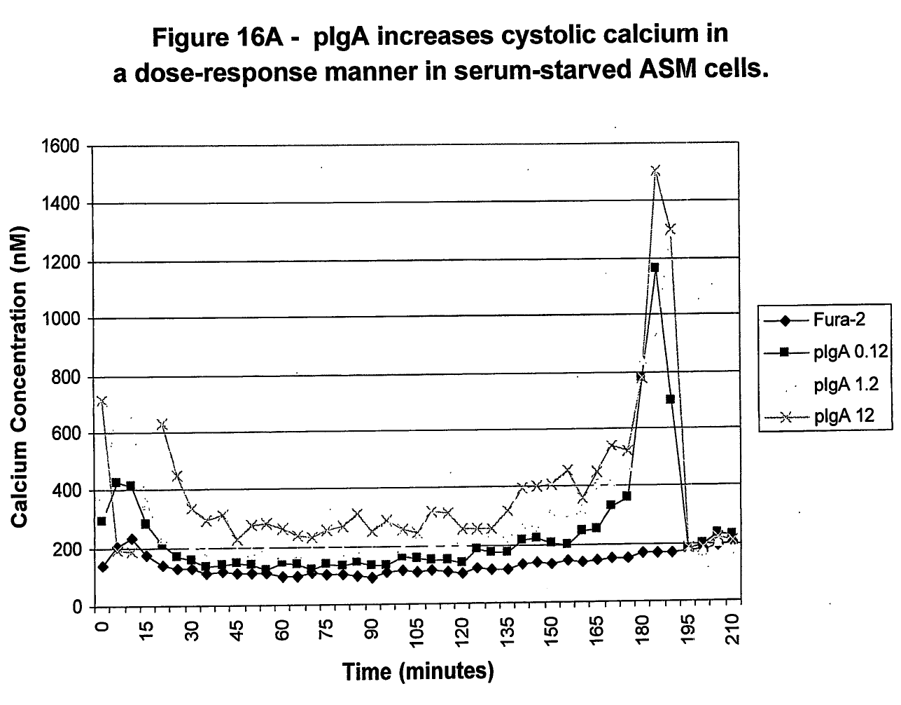

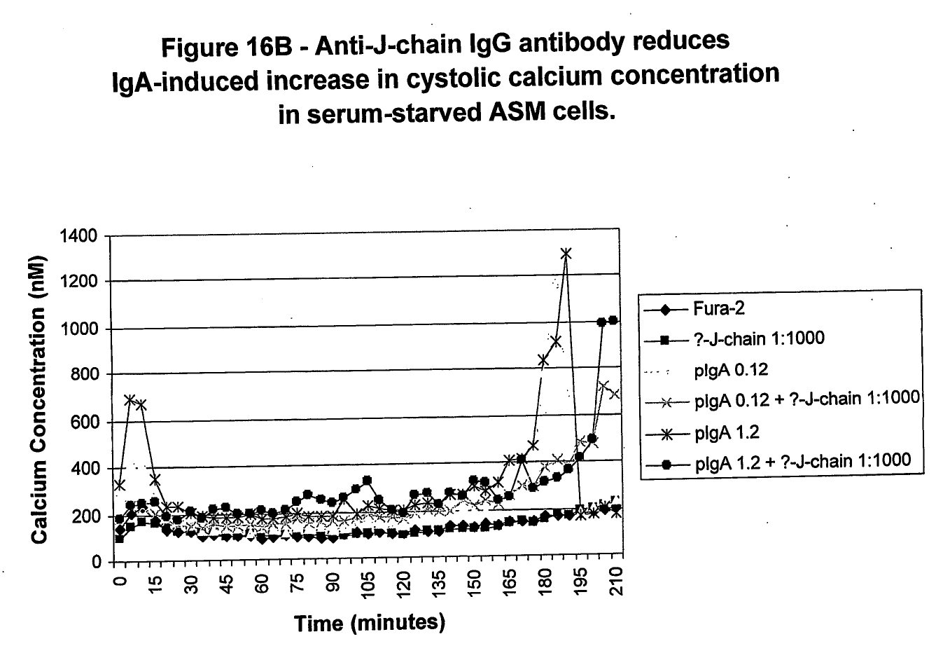

In another study, the effect of increasing concentrations of pIgA (0.12-12 μg/ml, in triplicate) was studied over 3 hours in serum-starved ASM grown in 96-well fluorescence plates for up to 10 days. These results are shown in FIG. 16A. These methods were also used to study the effect of a mouse monoclonal IgG1 antibody to J-chain 1 μg/ml (InnoGenex) on pIgA-induced changes in cytosolic calcium in serum-starved human ASM. These studies were undertaken to confirm that the effect of IgA is indeed mediated by ASM pIgR because the pIgR requires the presence of J-chain on polymeric immunoglobulins for efficient binding and endocytosis (Johansen et al, 2001). For this study, the ASM cells were treated in triplicate for 3 h at 37 C with (1) media alone, (2) mouse IgG1 anti-J-chain monoclonal antibody (InnoGenex) 1 μg/ml, (3) pIgA 0.12 μg/ml, (4) pIgA 0.12 μg/ml and anti-J-chain antibody, (5) pIgA 1.2 μg/ml, and (6) pIgA 1.2 μg/ml and anti-J-chain antibody (data shown in FIG. 16B).

(2) Calcium imaging. Fluorescence spectrophotometric and imaging techniques were used to study calcium signaling in Fura-2 loaded primary human ASM grown to 70% confluence on collagen-coated coverslips and serum-starved for 10-14 days. Cells were loaded with Fura-2 (Molecular Probes) for 1 h at 37 C. Cells were monitored for X min to ensure imaging stability, prior to being perfused with either (1) HEPES buffered KREBS solution containing 140 mM NaCl, 4.9 mM KCl, 1.4 mM KH₂PO₄, 1.2 mM MgCl₂, 11 mM Glucose, 25 mM HEPES, 2 mM CaCl₂ at pH 7.4 and 37 C; (2) pIgA (purchased from Dr. Vaerman) 0.12-12 μg/ml diluted in the KREBS buffer; or (3) mIgA (Pierce) 12 μg/ml in the KREBS buffer. Each cell was circled for automatic data processing of intracellular free calcium concentration for each cell and images were stored. Data was analyzed by Excel.

Results

Primary ASM Cell Cultures Express IgA Receptors by Immunofluorescence.

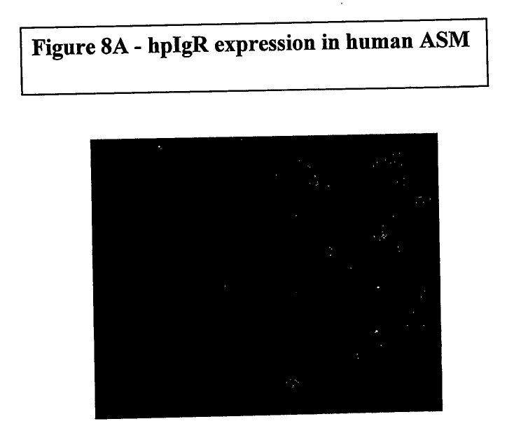

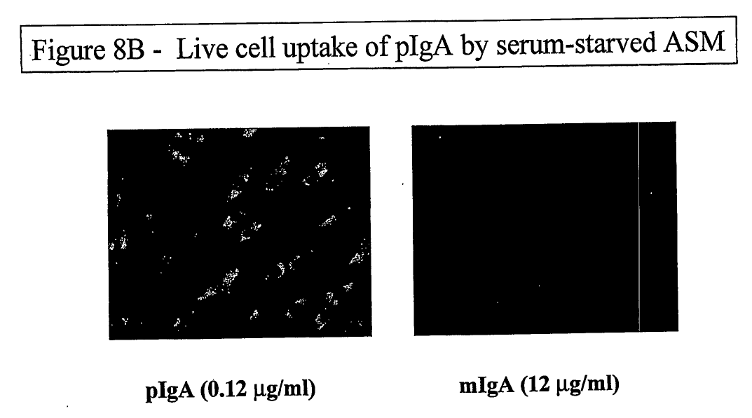

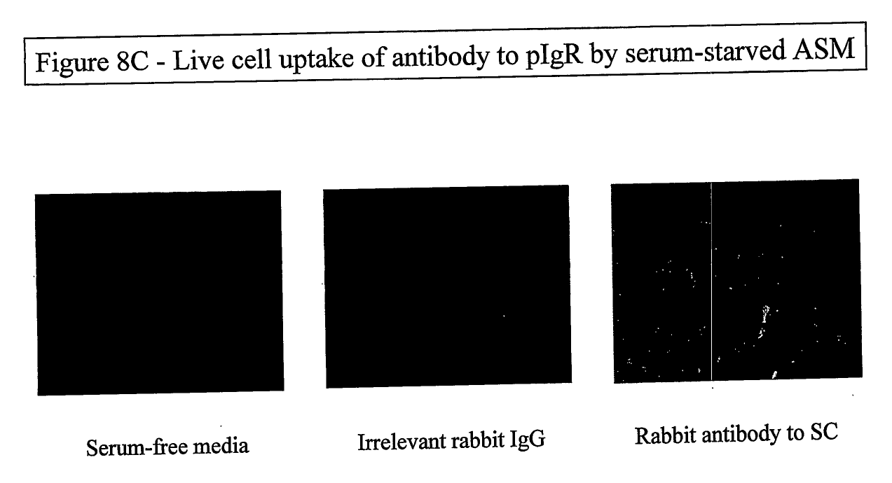

Human bronchial ASM cells from 3 different, non-asthmatic donors were obtained from Biowhitaker/Clonetics. All cells were used within 9 passages, and characterized by positive immunostaining with smooth muscle-specific actin and myosin, but not with factor VIII which recognizes endothelial cells. Primary cell cultures of serum-starved human bronchial ASM grown on coverslips were fixed and stained for pIgR with antibody to human SC, the extracellular portion of pIgR. FIG. 8A confirms green staining for pIgR in one representative ASM sample from the 3 different human. In another experiment, serum-starved ASM were incubated overnight with pIgA or mIgA, washed extensively, fixed with paraformaldehyde and then immuno-stained with antibodies to the alpha chain of IgA. Only ASM incubated with pIgA immunostained for the alpha chain (FIG. 8B, left panel), in contrast to ASM incubated with mIgA which does not bind pIgR (FIG. 8B, right panel). Live uptake experiments were also performed on serum-starved ASM pre-incubated with media alone (negative control), rabbit antibody to SC (ligand for pIgR), or an irrelevant rabbit IgG (control antibody). The cells were washed, fixed and stained with a FITC-conjugated secondary antibody to rabbit IgG. FIG. 8C confirms live-uptake of the rabbit antibody to SC (right panel) in contrast to the irrelevant IgG (middle panel) or serum alone.

These results show that serum-starved ASM express the pIgR protein and that it is capable of selectively binding its ligands (pIgA or rabbit antibody to SC) and not irrelevant Iigands (mIgA or Irrelevant rabbit IgG).

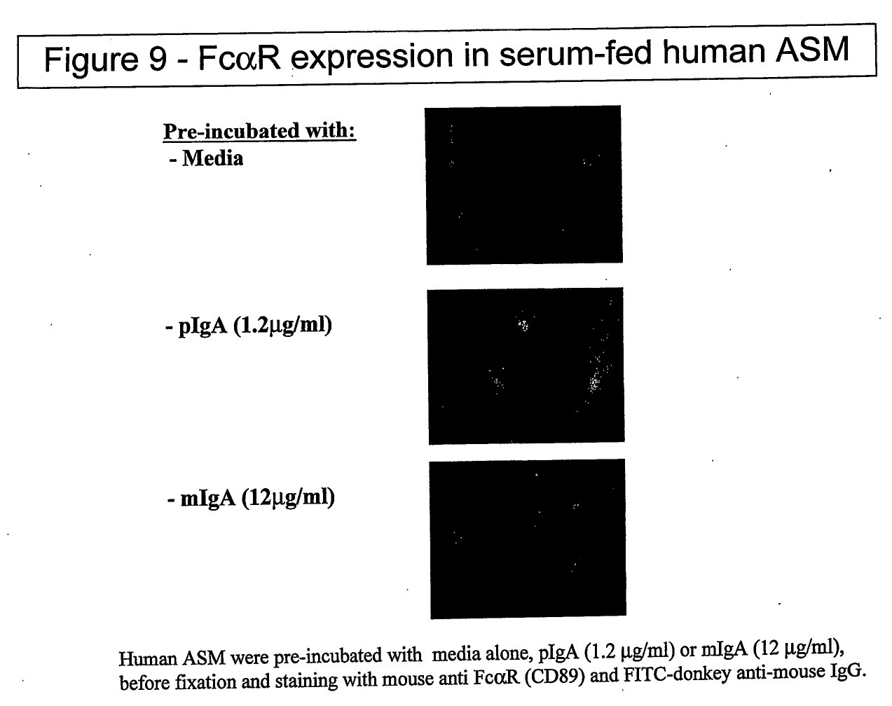

FIG. 9 shows that in contrast to serum-starved ASM, non-starving ASM do immunostain for the FcαR (top panel). In addition, this FcαR expression increases after pre-incubation of live cells with either pIgA (middle panel) or mIgA (lower panel), both of which can bind the FcαR. Thus FIGS. 8 and 9 confirm that ASM cells express both the pIgR and the FcαR protein. Furthermore, ligands to FcαR upregulate its expression on serum-fed ASM.

Primary Cultures of ASM have IgA Receptors Detected by Western Blotting.

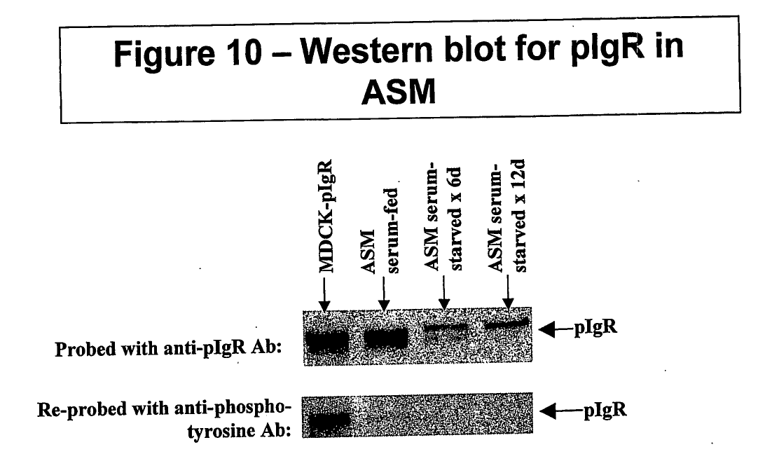

FIG. 10 shows a western blot that confirms the presence of pIgR in both serum-starved and non-starving ASM. MDCK-HpIgR epithelial cells were used as a positive control for pIgR protein expression. The results of these western blots confirmed the presence of a band consistent with pIgR (FIG. 10, upper panel). It is of interest that the pIgR band from the 12-day serum-starved ASM appeared as a singlet, in contrast to the pIgR band from transfected MDCK cells, which appears as a doublet due to phosphorylation of the receptor. Non-starving ASM expressed the doublet pIgR band, as do the 6-day starving cells (FIG. 10, upper panel). Stripping this blot and re-probing with 4G10 (UBI) monoclonal antibody to phospho-tyrosine shows that the second band of pIgR is phosphorylated in non-starving ASM and in transfected MDCK cells, as commonly found with many receptors (FIG. 10, lower panel).

FIG. 10 adds further evidence that ASM express pIgR protein and that it appears as a doublet due to phosphorylation when the cells are grown with serum.

Primary Cultures of ASM have IgA Receptors Detected by RT-PCR

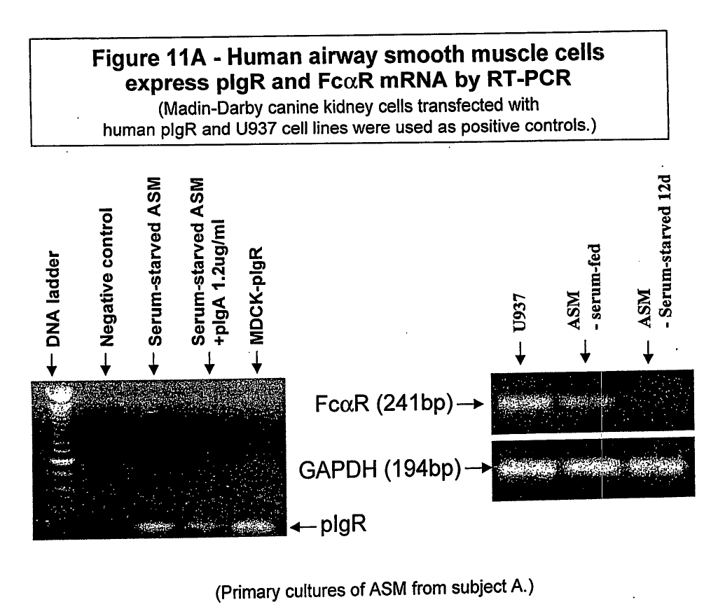

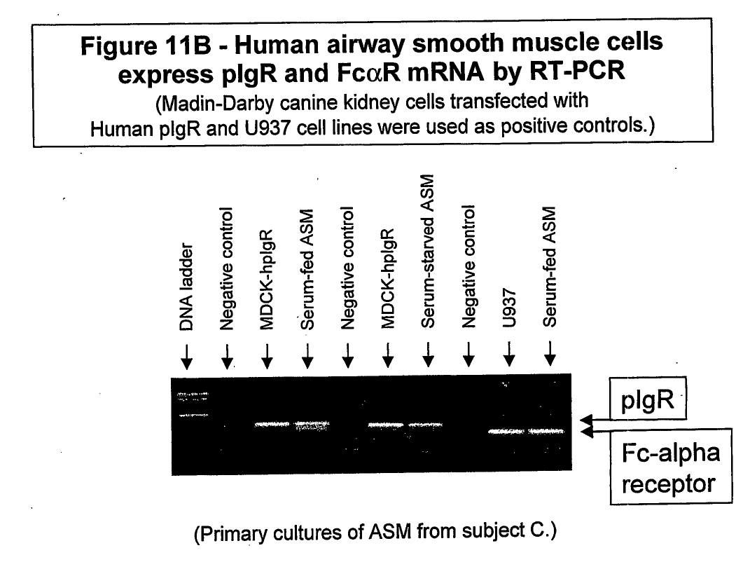

FIGS. 11A and 11B show that ASM have mRNA encoding the cytoplasmic domain of pIgR by RT-PCR. MDCK-HpIgR cells were used as a positive control for pIgR mRNA message. DNA sequencing of this band (339 bp) revealed sequence homology to the epithelial pIgR. These results confirm that ASM express mRNA for pIgR.

FIG. 11A also shows that serum-starved ASM do not have mRNA encoding the IgA-binding domain of FcαR by RT-PCR (right lane of image on right). In contrast, RT-PCR with serum-fed cells show that ASM do express FcαR (FIG. 11A, middle lane of image on right; and FIG. 11B). U937 cells were used as a positive control for FcαR mRNA message. DNA sequencing of the RT-PCR product from non-starving ASM (241 bp) confirmed it is the FcαR.

These results show that ASM express mRNA for both pIgR and FcαR. Furthermore, serum-deprivation down-regulates mRNA expression for FcαR in ASM.

Polymeric IgA Increases Cytosolic Calcium Concentrations in ASM.

Incubation with pIgA significantly increased cytosolic calcium concentrations in 8 day serum-starved ASM in contrast to mIgA or media alone (Table 2). The effect of pIgA was seen after 1 h, peaked at 1 h 15 min and was sustained to 1.5 h, which was the duration of the experiment. In another experiment, increasing concentrations of pIgA caused an increased rise in cytosolic calcium in serum-starved ASM (FIG. 16B). The peak effect was observed in these cells after 2 h and the experiment was continued for a total of 3 h. These results confirm that pIgA increases cytosolic calcium in a dose-response manner.

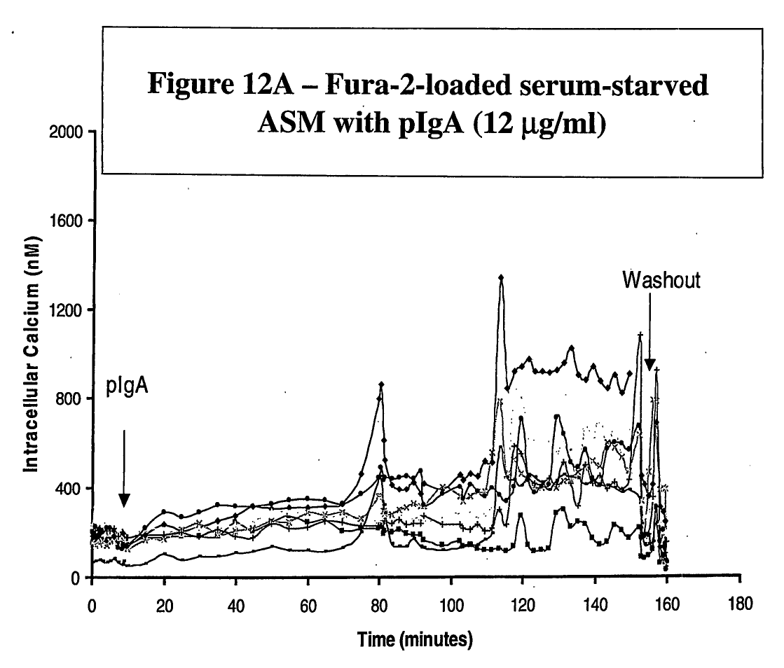

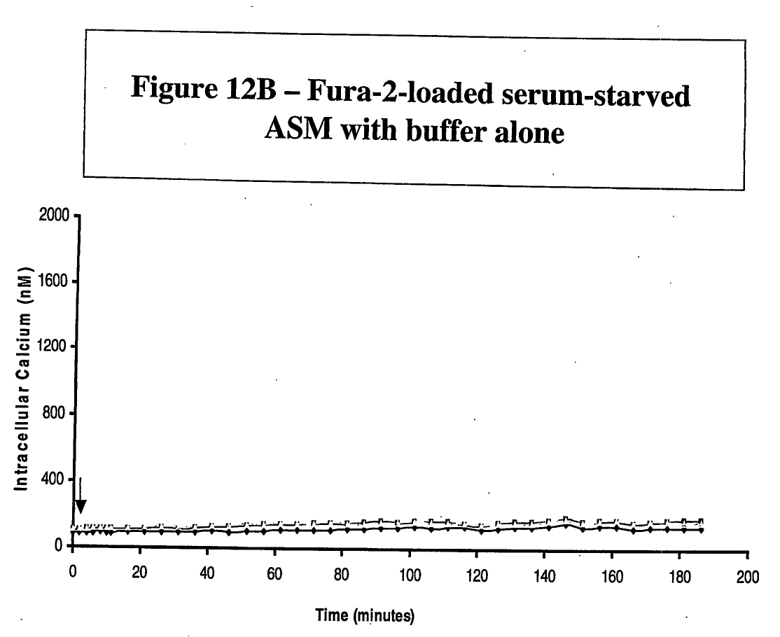

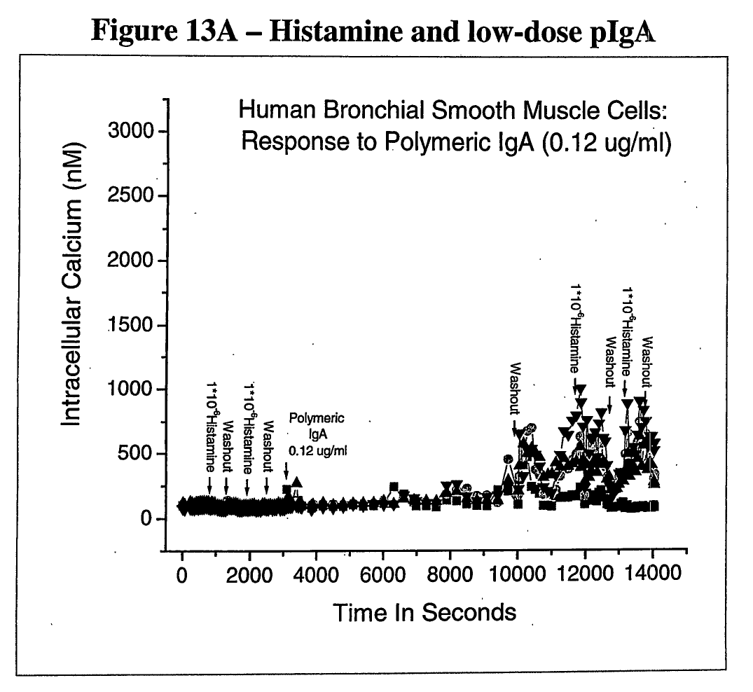

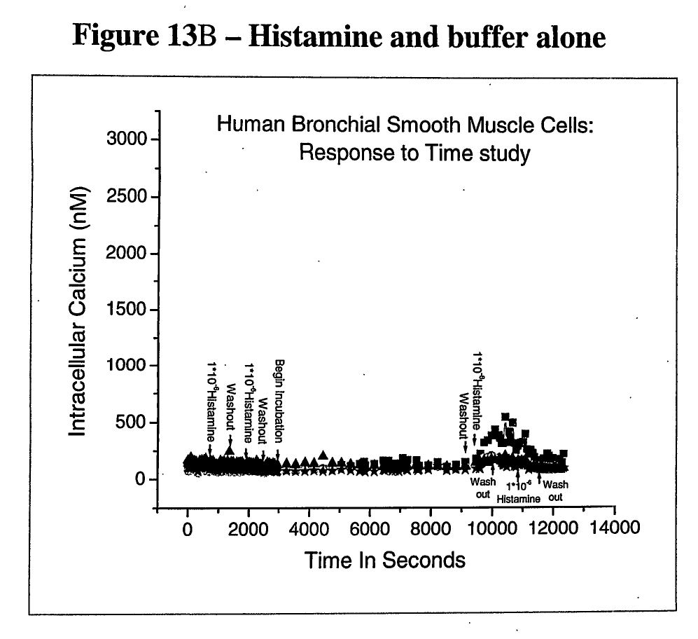

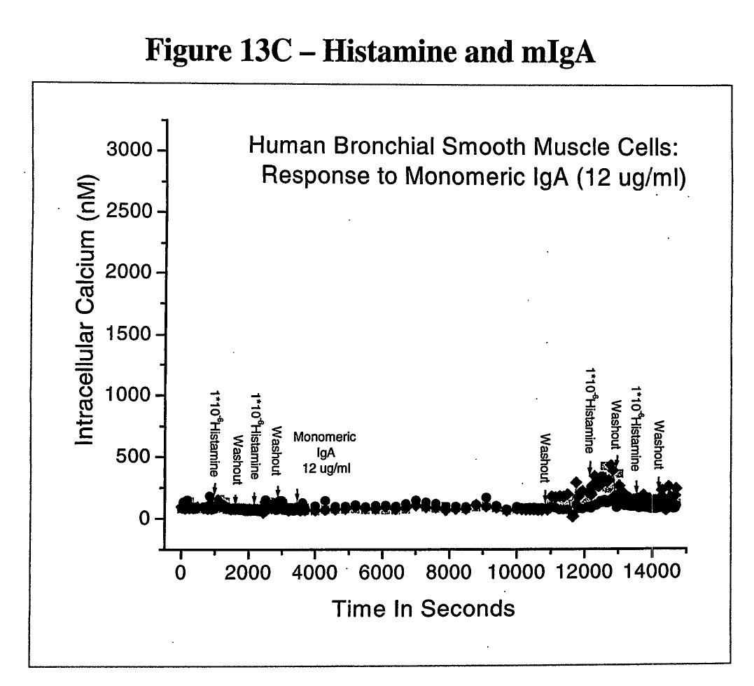

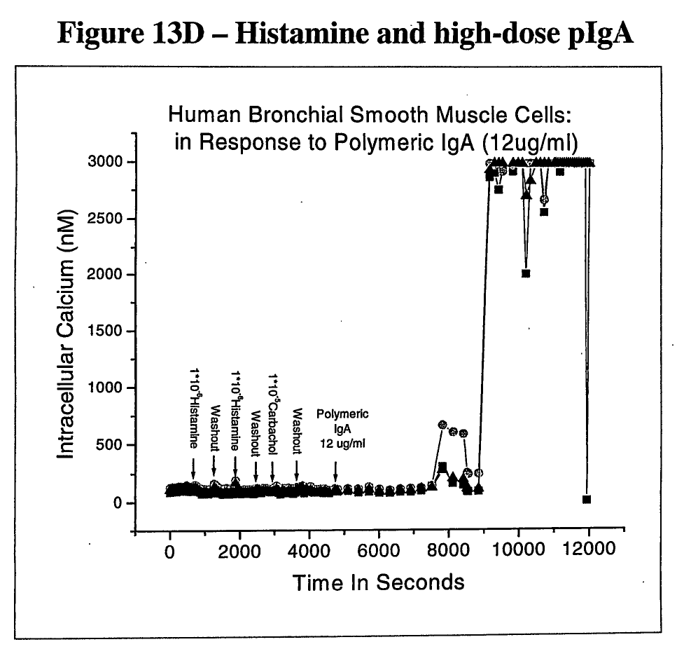

Fluorescence spectrophotometric and imaging techniques confirmed these results. Incubation of serum-starved ASM with pIgA significantly increased cytosolic calcium concentrations (FIG. 12A), in contrast to incubation with buffer alone which shows no change in calcium (FIG. 12B). In FIGS. 12 and 13, each line represents a different cell monitored for the duration of the experiment on the same day. Each graph represents one of 3-5 studies per condition. Of greatest interest, the pIgA-induced rise in cytosolic calcium concentrations occurred consistently after about 1.5 h and manifested a sustained response with an oscillating pattern for the duration of the experiment.

Similar experiments were done after first testing the serum-starved ASM with histamine, a known stimulus for ASM contraction and bronchoconstriction. After the histamine was washed off, a low concentration of pIgA (0.12 μg/ml) was added. After 1 h, cytosolic calcium concentrations increased and rose 4-6 fold with repeat histamine stimulation after washing off pIgA (FIG. 13A). The time control experiment in FIG. 12B shows that pre-exposure to histamine sensitizes the serum-starved ASM cells to a second histamine exposure 2 h later with less than a 2-fold increase in intracellular calcium concentration. When a similar experiment was done with a high dose of mIgA (12 μg/ml), re-exposure to histamine increased calcium levels less than 2-fold after washing of the mIgA (FIG. 13C), as occurred with buffer alone in FIG. 13B. Finally, repeating this experiment with a high dose of pIgA (12 μg/ml) after initial exposure to histamine and carbachol (another stimulus for ASM contraction and bronchoconstriction), caused a dramatic rise in cytosolic calcium concentrations beginning 50 minutes later and going off-scale at 66 minutes with over a 30-fold increase (FIG. 13D).

These results show a unique response of ASM to pIgA that has never been described before. First, pIgA causes a consistent delayed rise in cytosolic calcium concentrations. This delayed response provides a window of opportunity to potentially alter cell signaling events triggered by the rise in cytosolic calcium concentrations. Second, pIgA causes a sustained rise in calcium concentrations that oscillate. The frequency of these oscillations have been associated with increasing the activity of the pro-inflammatory transcription factor, NFcκB (Hu et al, J. Biol. Chem. 274:33995-33998, 1999). Chronic airway inflammation characterizes asthma and has been associated with airway remodeling that leads to irreversible changes. Third, histamine appears to sensitize ASM to pIgA. This particular finding potentially has major implications for treatment of allergic asthmatics who develop upper airway infections. Fourth, the lack of response to mIgA and absence of FcαR in non-starving ASM indicate that pIgA mediates its effect via pIgR.

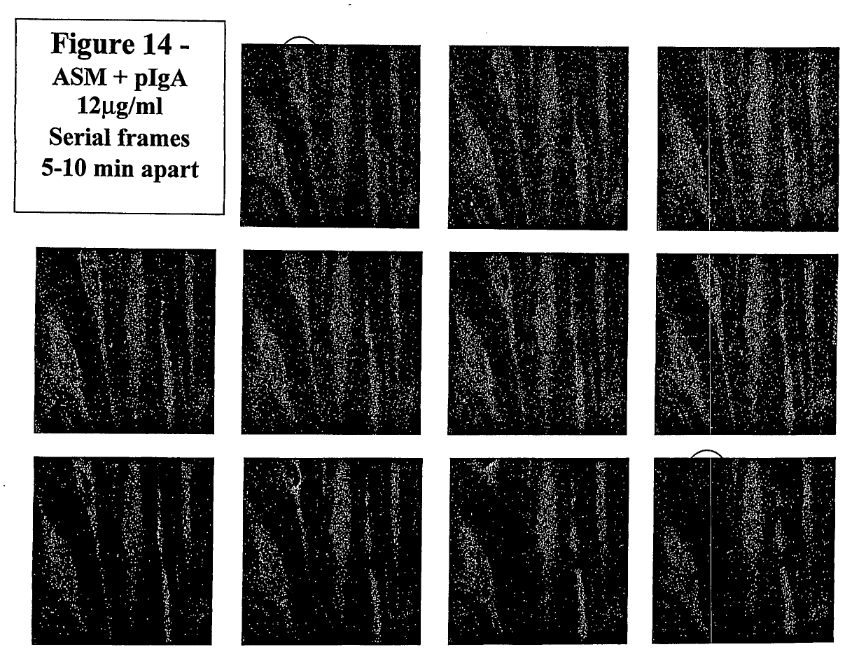

Finally, imaging of serum-starved ASM exposed to the highest concentration of pIgA (12 μg/ml) during the Fura-2 calcium experiments shows ASM contraction (FIG. 14). The cell that is circled in Frame 1 contracts and disappears by Frame 11. Not all the cells respond at the exact same time or to the same degree. However, even the cell in the lower right hand corner also changes shape. These cells were grown on collagen coverslips and were noticeably subconfluent, and may account for the differences in time and intensity of response. Cell contraction has never been described before in response to pIgA. The fact that this happens in serum-starved cells, again, implies that this pIgA effect is mediated via pIgR.

### Example 4

The inventors have shown that anti-j chain antibody (commercially available mouse monoclonal IgG1) decreases pIgA-induced increase in. cytosolic Ca++ (pIgA 1.2 and 0.12 ug/ml) in Fura-2 loaded primary cultures of serum-starved human airway smooth muscles grown on fluorescence plates (results are shown in FIG. 16B). This data shows that inhibiting the binding of pIgA to mesenchymal pIgR by, for example, targeting J-chain, modulates calcium responses.

### Example 5

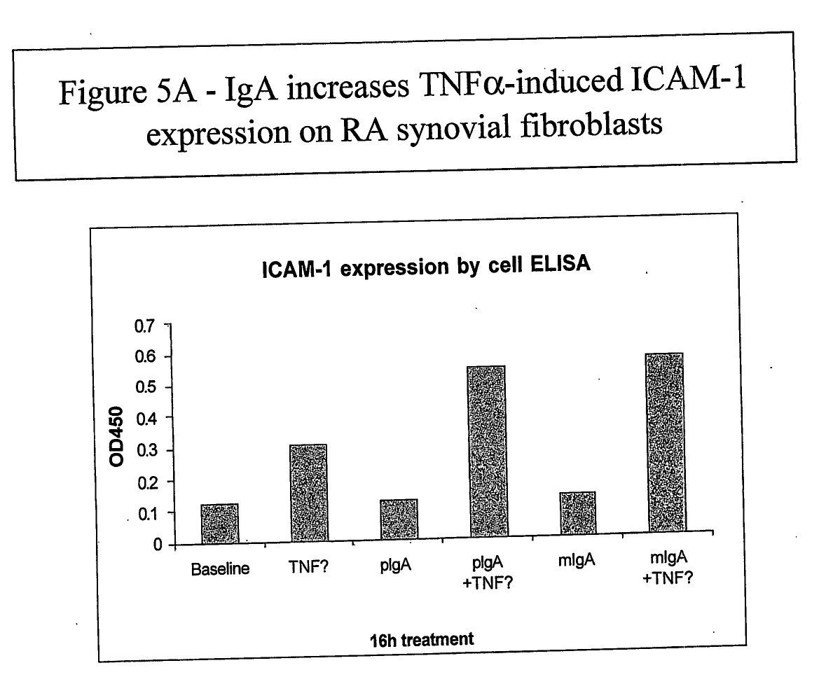

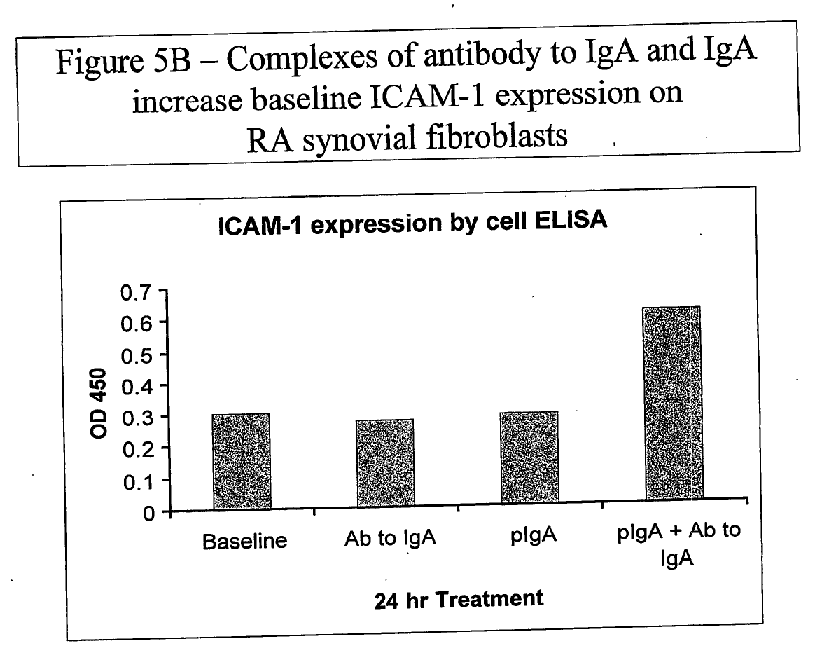

The inventors have studied the downstream effects of adding IgA to osteoarthritis and rheumatoid arthritis synovial fibroblasts. In one experiment, the inventors demonstrated that IgA increased NFkB DNA binding in both RA and OA synovial fibroblasts (FIG. 4). In another experiment, the inventors demonstrated that IgA increased TNF-alpha-induced ICAM-1 expression by cell ELISA (see FIG. 5A). In additional experiments, the inventors demonstrated that IgA increased ICAM-1 expression in RA synovial fibroblasts by cell ELISA only in the presence of an antibody to the alpha chain of IgA thereby forming an immune complex like a pseudo-rheumatoid factor (FIG. 5B).

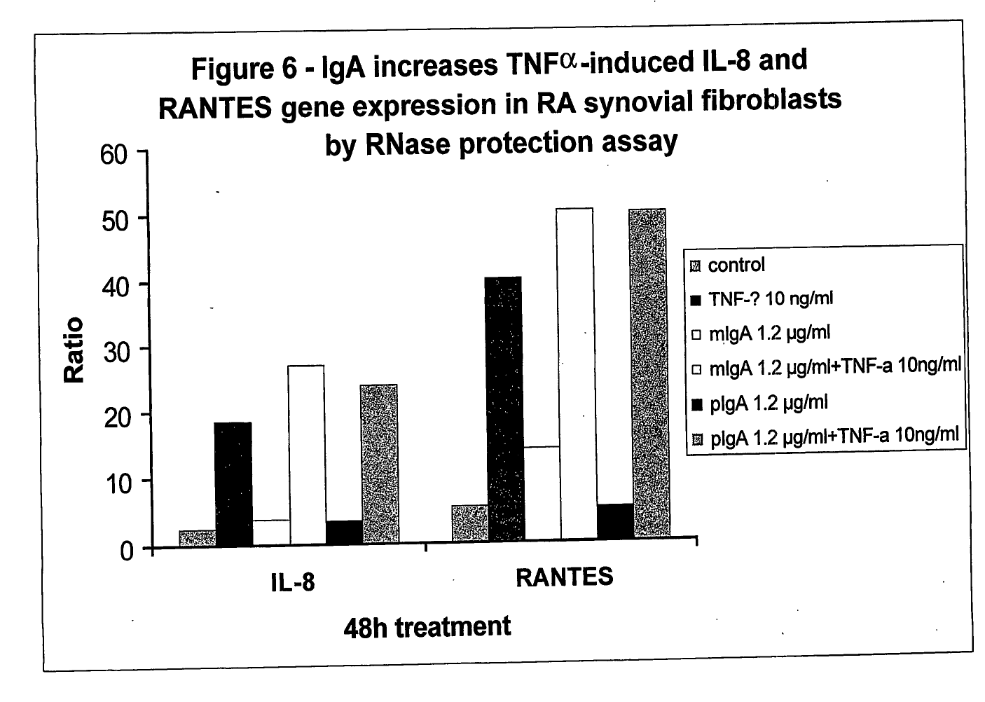

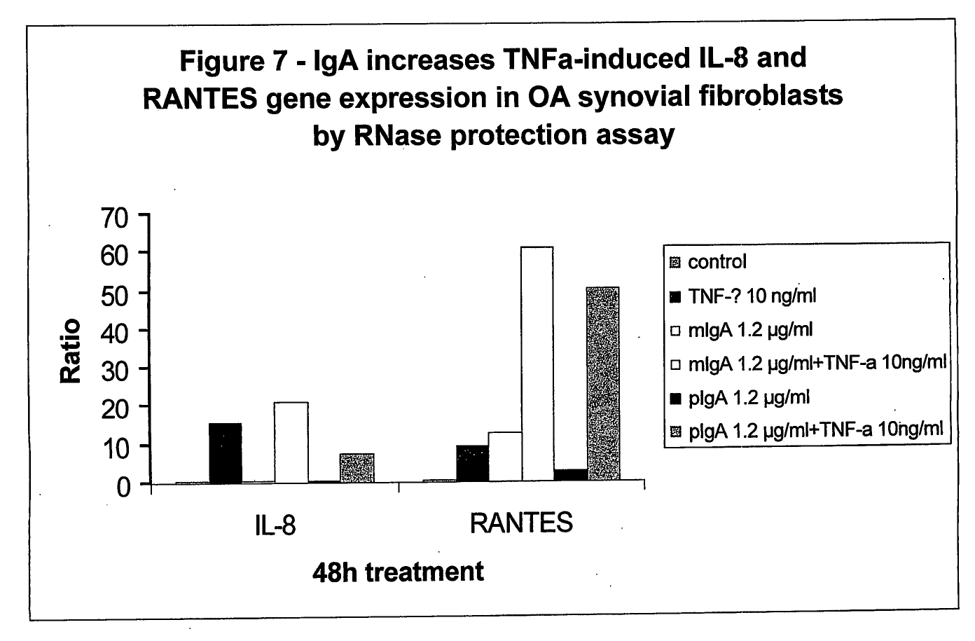

The inventors also demonstrated that IgA increases TNFα-induced IL-8 and RANTES gene expression in synovial fibroblasts from patients, with both rheumatoid arthritis (FIG. 6) and osteoarthritis (FIG. 7) by RNase protection assay.

Methods

ICAM-1 Protein Expression was Evaluated Using a Cell ELISA Method.

Primary cultures of RA and OA synovial fibroblasts were cultured on 96 well plates until confluent. To study the effect of IgA and TNFα, cells were incubated for 16 h with the following conditions in triplicate: (1) cell culture media alone (baseline ICAM-1 expression); (2) TNFα 10 ng/ml (positive control); (3) pIgA 1.2 μg/ml; (4) pIgA 1.2 μg/ml and TNFα 10 ng/ml ; (5) mIgA 1.2 μg/ml; and (6) mIgA 1.2 μg/ml and TNFα 10 ng/ml. To study the effect of an immune complex containing IgA, cells were treated for 24 h with (1) media alone (baseline expression); (2) goat polyclonal antibody to the alpha chain of IgA 2.4 μg/ml (Jackson Laboratories); (3) pIgA 1.2 μg/ml; and (4) pIgA mixed together with the antibody to IgA. Cells were washed and then fixed with 4% paraformaldehyde for 30 minutes on ice, and blocked with 2% bovine serum albumin in PBS at 37 C. Mouse anti-human ICAM-1 antibody (Sigma) diluted 1:500 in 1% BSA in PBS was added to the cells for 1 hour at 37° C., followed by goat anti-mouse HRP-conjugate IgG (1:5000) (Bio-Rad, Hercules Calif.) for 30 min at 37 C. Plates were developed with the HRP substrate, TMB (Sigma) and the color change was stopped with 0.18M H₂SO₄. The absorbance at OD450 was measured on a Spectramax 190 from Molecular Devices utilizing SOFTmax Pro software.

The effect of IgA on gene expression of inflammatory mediators was determined by RNAse protection assay using a custom-made multi-probe template set purchased from BD Biosciences that included probes for IL-8 and RANTES as well as housekeeping probes for L32 and GAPDH. Radioactive labelling for the RNase protection assays was performed according to the manufacturer's directions. RA and OA synovial fibroblasts were incubated for 48 h with (1) cell culture media alone (baseline expression); (2) TNFα long/ml (positive control); (3) pIgA 1.2 μg/ml; (4) pIgA 1.2 μg/ml and TNFα long/ml; (5) mIgA 1.2 μg/ml; and (6) mIgA 1.2 μg/ml and TNFα long/ml. The autoradiograph was scanned with a phosphorimager to detect band intensities. Gene expression was quantitated as the ratio of the band intensity of the gene of interest to the intensity of a housekeeping gene.

### Example 6

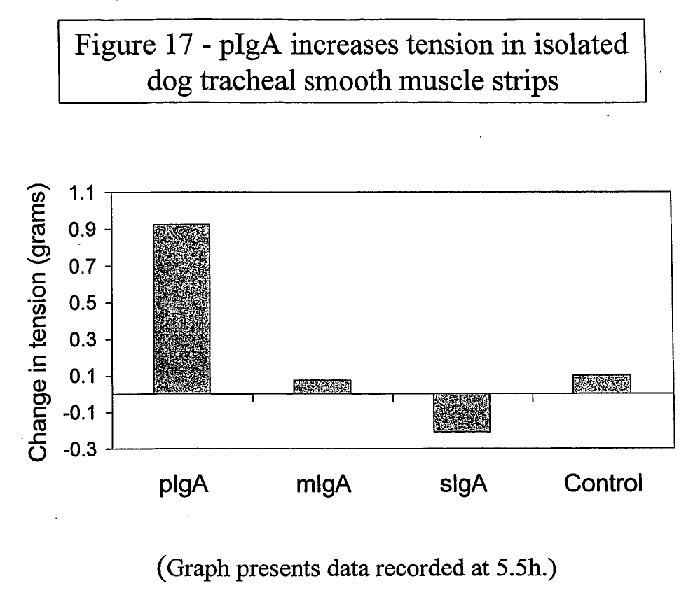

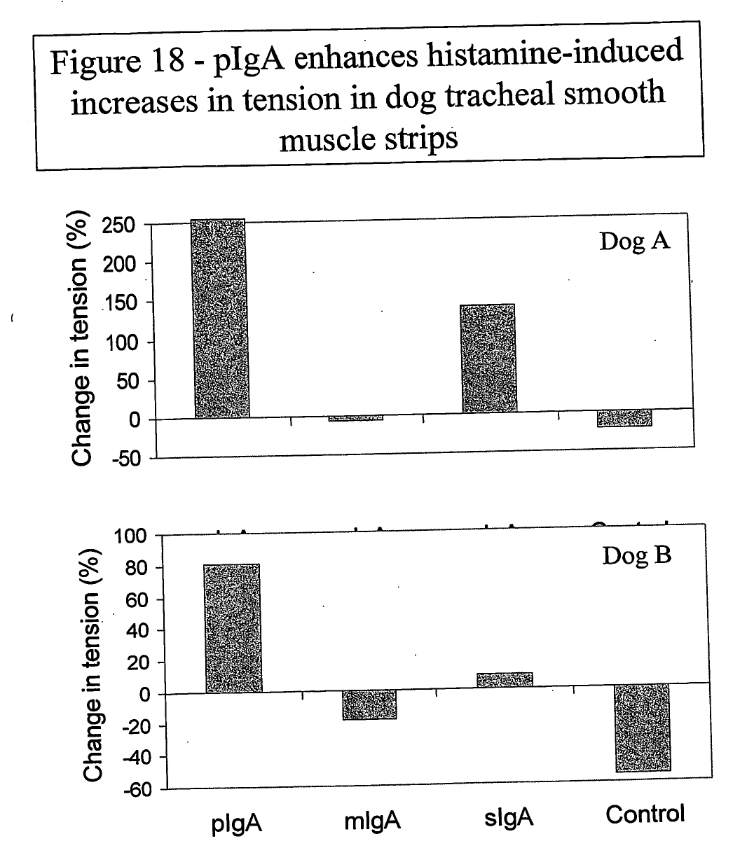

The inventors have also shown that: (a) pIgA increases tension ex vivo in dog tracheal smooth muscle strips in contrast to no change with mIgA (does not bind pIgR) or with control buffer (FIG. 17) and (b) pre-incubation of these strips with pIgA enhances histamine-induced increase in tension (FIG. 18). These ex-vivo experiments with dog tissue confirm the in vitro cytosolic calcium studies obtained with human ASM: not only does pIgA modulate cytosolic calcium and ASM contraction, but binding to pIgR also. sensitizes the smooth muscle cells to non-specific contractile agonists like histamine.

Tension experiments were done using canine tracheal smooth muscle strips using methodology similar to Antonissen et al (1980). Roughly equal lengths of dog trachealis smooth muscle strips (˜1 by 10 mm) were mounted on a tissue tension apparatus that was calibrated so that 1 g of tension was reflected by two large boxes on the strip chart recorder's trace paper. The smooth muscle strips were perfused with warmed oxygenated KREBS-Henseleite buffer solution. Once the tissue was connected to the transducer hook, roughly 1 gm of passive tension was added to obtain the optimal alignment of actin and myosin within the smooth muscle cells. The effect of different isoforms of IgA on tension was studied by incubating the strips for 16 hours at 37 C with (1) buffer alone (control); (2) human mIgA (4.6 mg/ml); (3) human pIgA (12 mg/ml); and (4) human sIgA (4.6 mg/ml). Results were recorded as the net change in tension (gm). FIG. 17 shows the change in tension obtained at 5.5 h into the incubation period. This time-point reflects peak tension change.

For studies evaluating the effect of IgA on histamine-induced contraction, dog tracheal smooth muscle strips were mounted on the tissue tension apparatus and the tension was normalized in mN (=force/cm²). The responsiveness of the tissue samples to 80 mM KCl (to test for muscle viability and health) as well as to histamine 20 mM (a non-specific bronchoconstrictor) was determined. Each strip was incubated for 4 h at 37 C with: (1) buffer alone (control); (2) human mIgA (2.3 mg/ml); (3) human pIgA (6 mg/ml); or (4) human sIgA (2.3 mg/ml). A lower dose of pIgA was used so as to cause a minimal change in tissue tension. After washing off the test conditions, the ability of each strip to contract in response to histamine 20 mM was repeated. After washing again, the response to KCl was re-tested. The length and weight of each muscle strip was measured to normalize tension measurements. FIG. 18 shows the percent change in histamine response before the incubation period as compared to after 4 h treatment with the IgA isoforms.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1

| | |
|---|---|
| Protein | Primer sequences |

| | |
|---|---|
| plgR: cytoplas- | |
| mic domain | |
| sense | 5′ GAG CCC ACT CCC TGC TCT AAC 3′ |
| antisense | 5′ AGA AGA GGG GAA GGA CGG GAG 3′ |
| FcαR: IgA bind- | |
| ing domain | |
| sense | 5′ CCT CAG TCT GGG GCT TTC TTT 3′ |
| antisense | 5′ CTT GTT TGC GTC CAT GTG GTC 3′ |

TABLE 2
Cytosolic calcium concentration (nM) in serum-starved ASM at
100 min after adding stimulus:

| Expt. | control | mlgA | plgA*# |
|---|---|---|---|
| 1 | 353.50 | 374.00 | 1556.00 |
| 2 | 501.46 | 1961.61 | 2481.45 |
| 3 | 640.25 | 853.74 | 556.78 |
| 4 | 537.25 | 995.72 | 1465.87 |
| 5 | 445.43 | 588.41 | 1391.39 |
| means | 495.58 | 954.70 | 1490.30 |
| S.E. | 47.56 | 273.12 | 305.12 |

T-TEST
P = 0.08, mlgA versus control
*P = 0.02 (P < 0.05), plgA versus control
#P = 0.046 (P < 0.05), plgA versus mlgA

### FULL CITATIONS FOR REFERENCES REFERRED TO IN THE SPECIFICATION

Ackermann L W, Wollenweber L A, Denning G M. IL-4 and IFN-γ increase steady state levels of polymeric Ig receptor mRNA in human airway and intestinal epithelial cells. J. Immunol. 162:5112-5118, 1999.

Albrechtsen M, Yeaman G R, Kerr M A. Characterization of the IgA receptor from human polymorphonuclear leucocytes. Immunology. 64:201-5, 1988.

Antonissen L A, Mitchell R W, Kroeger E A, Kepron W, Stephens N L, Bergen J. Histamine pharmacology in airway smooth muscle from a canine model of asthma. J Pharmacol Exp Ther 213:150-155, 1980.

Bell D Y, Haseman J A, Spock, A, McLennan G, Hook G E R. Plasma proteins of the bronchoalveolar surface of the lungs of smokers and nonsmokers. Am. Rev. Respir. Dis. 124: 72-79, 1981.

Childers N K, Bruce M G, McGhee J R. Molecular mechanisms of Immunoglobulin A defense. Ann. Rev. Microbiol. 43: 503-536, 1989.

Choy E H S and Panayi G S. Cytokine pathways and joint inflammation in rheumatoid arthritis. N. Engl. J. Med. 344:907-916, 2001.

Davies D E, Wicks J, Powell R M, Puddicombe S M, Holgate S T. Airway remodeling in asthma: new insights. J. Allergy Clin. Immuno. 111:215-25, 2003.

Deo Y M, Sundarapandiyan K, Keler T, Wallace P K, Graziano R F. Bispecific molecules directed to the Fc receptor for IgA (Fc alpha RI, CD89) and tumor antigens efficiently promote cell-mediated cytotoxicity of tumor targets in whole blood. J Immunol. 160:1677-86, 1998.

Gioud-Paquet M, Auvinet M, Raffin T, Girard P, Bouvier M, Lejeune E, Monier J C. IgM rheumatoid factor (RF), IgA RF, IgE RF, and IgG RF detected by ELISA in rheumatoid arthritis. Ann. Rheum. Dis. 46:65-71, 1987.

Halayko A J, Salari H, Ma X, Stephens N L. Markers of airway smooth muscle cell phenotype. Am. J. Physiol. 270:L1040-51, 1996.

Halayko A J, Camoretti-Mercado B, Forsythe S M, Vieira J E, Mitchell R W, Wylam M E, et al. Divergent differentiation paths in airway smooth muscle culture: induction of functionally contractile myocytes. Am. J. Physiol. 276:L197-206, 1999.

Holgate S T, Davies D E, Lackie P M, Wilson S J, Puddicombe S M, Lordan J L. Epithelial-mesenchymal interactions in the pathogenesis of asthma. J. Allergy Clin. Immunol. 105:193-204, 2000.

Houssien D A, Jonsson T, Davies E, Scott D L. Clinical significance of IgA rheumatoid factor subclasses in rheumatoid arthritis. J. Rheumatol. 24:2119-22, 1997.

Hu Q, Deshpande S, Irani K, Ziegelstein R C. [Ca2+]ᵢ oscillation frequency regulates agonist-stimulated NF-KB transcriptional activity. J. Biol. Chem. 274:33995-33998, 1999.

Jenkins J K, Hardy K J, McMurray R W. The Pathogenesis of rheumatoid arthritis: a guide to therapy. Am. J. Med. Sci. 323:171-180, 2002.

Johansen F E, Braathen R, Brandtzaeg P. The J chain is essential for polymeric Ig receptor-mediated epithelial transport of IgA. J Immunol. 167:5185-92, 2001.

Jonsson T, Valdimarsson H. Clinical significance of rheumatoid factor isotypes in seropositive arthritis. Rheumatol. Int. 12:111-3, 1992.

Jonsson T, Arinbjarnarson S, Thorsteinsson J, Steinsson K, Geirsson A J, Jonsson H, Valdimarsson H. Raised IgA rheumatoid factor (RF) but not IgM RF or IgG RF is associated with extra-articular manifestations in rheumatoid arthritis. Scand. J. Rheumatol. 24:372-5, 1995.

Jonsson T, Steinsson K, Jonsson H, Geirsson A J, Thorsteinsson J, Valdimarsson H. Combined elevation of IgM and IgA rheumatoid factor has, high diagnostic specificity for rheumatoid arthritis. Rheumatol. Int. 18:119-22, 1998.

Jorgenssen C, Legouffe M C, Bologna C, Brochier J, Sany J. IgA isotype rheumatoid factor in rheumatoid arthritis: clinical implications. Clin. Exp. Rheumatol. 14:301-4, 1996.

Kerr M A, Woof J M. Fc-alpha Receptors. In: Mucosal Immunology. P L Ogra, J Mestecky, M Lamm, W Strober, J Bienenstock, J R McGhee, editors. San Diego; Academic Press, 1999.

Louis R, Shute J, Biagi, S, Stanciu L, Marrelli F, Tenor H, Hidi R, Djukanovic R. Cell infiltration, ICAM-1 expression, and eosinophil chemotactic activity in asthmatic sputum. Am. J. Respir. Crit. Care Med. 155:466-72, 1997.

MacKenzie S J, Kerr M A. IgM monoclonal antibodies recognizing FcaR but not FcgRIII trigger a respiratory burst in neutrophils although both trigger an increase in intracellular calcium levels and degranulation. Biochem J 306:519-523, 1995.

Maliszewski C R, March C H, Schoenborn M A, Gimpel S, Shen L. Expression cloning of a human Fc receptor for IgA. J. Exp. Med. 172:1665-1672, 1990.

Message S D, Johnston S L. Viruses in asthma. Br Med Bull 61:29-43, 2002.

Mestecky J, Moro I, Underdown B J. Mucosal Immunoglobulins. In: Mucosal Immunology. P L Ogra, J Mestecky, M. Lamm, W Strober, J Bienenstock, J R McGhee, editors. San Diego: Academic Press, 1999.

Monteiro R C, Kubagawa H, Cooper M D. Cellular distribution, regulation, and biochemical nature of an Fc alpha receptor in humans. J Exp Med. 171:597-613, 1990.

Morton H C, van den Herik-Oudijk I E, Vossebeld P, Snijders A, Verhoeven A J, Capel P J, van de Winkel J G. Functional association between the human myeloid immunoglobulin A Fc receptor (CD89) and FcR gamma chain. Molecular basis for CD89/FcR gamma chain association. J Biol Chem. 270:29781-7, 1995.

Morton H C, Van Egmond M, Van de Winkel J G. Structure and function of human IgA Fc receptors (FαR). Crit. Rev. Immunol. 16: 423-440, 1996.

Mostov K E, Altschuler Y, Chapin S J, Enrich C, Low S H, Luton F, Richman-Eisenstat J, Singer K, Tang K, Weimbs T. Regulation of protein traffic in polarized epithelial cells: the pIgR model. Cold Spring Harbor Symposia on Quantitative Biology. Protein Kinesis: The Dynamics of Protein Trafficking and Stability. 60: 775-781, 1995.

Mostov K, Kaetzel C S. Immunoglobulin transport and the polymeric immunoglobulin receptor. In: Mucosal Immunology. P L Ogra, J Mestecky, M. Lamm, W Strober, J Bienenstock, J R McGhee, editors. San Diego: Academic Press, 1999.

Nahm D H, Kim H Y, Park H S. Elevation of specific immunoglobulin A antibodies to both allergen and bacterial antigen in induced sputum from asthmatics. Eur. Respir. J. 12:540-5, 1998.

Nahm D H, Park H S. Correlation between IgA antibody and eosinophil cationic protein levels in induced sputum from asthmatic patients. Clin. Exp. Allergy 27:676-81, 1997.

Otten H G, Daha M R, van Laar J M, de Rooy H H, Breedveld F C. Subclass distribution and size of human IgA rheumatoid factor at mucosal and nonmucosal sites. Arthritis Rheum. 34:831-9, 1991.

Pai S, Pai L, Birkenfeldt R. Correlation of serum IgA rheumatoid factor levels with disease severity in rheumatoid arthritis. Scand. J. Rheumatol. 27: 252-6, 1998.

Patry C, Herbelin A, Lehuen A, Bach J F, Monteiro R C. Fc alpha receptors mediate release of tumour necrosis factor-alpha and interleukin-6 by human monocytes following receptor aggregation. Immunology. 86:1-5, 1995.

Piskurich J F, Blanchard M H, Youngman K R, France J A, Kaetzel C S. Molecular cloning of the mouse polymeric Ig receptor. Functional regions of the molecule are conserved among five mammalian species. J. Immuno. 154:1735-1747, 1995.

Schmidt D, Rabe K F. Immune mechanisms of smooth muscle hyperreactivity in asthma. J. Allergy Clin. Immunol. 105:673-82, 2000.

Schrohenloher R E, Koopman W J, Alarcon G S. Molecular forms of IgA rheumatoid factor in serum and synovial fluid of patients with rheumatoid arthritis. Arthritis Rheum. 29:1194-202, 1986.

Scott D L, Symmons D P, Coulton B L, Popert A J. Long-term outcome of treating rheumatoid arthritis: results after 20 years. Lancet 1:1108-11, 1987.

Sheets M D, Amersdorfer P. Finnern R, Sargent P, Lindquist E, Schier R, Hemingsen G, Wong C, Gerhart J C, Marks J D, Lindqvist E. Efficient construction of a large nonimmune phage antibody library: the production of high-affinity human single-chain antibodies to protein antigens. Proc Natl Acad Sci USA. 95:6157-62, 1998.

Shen L. A monoclonal antibody specific for immunoglobulin A receptor triggers polymorphonuclear neutrophil superoxide release. J Leukoc Biol 51: 373-378, 1992.

Stewart W W, Mazengera R L, Shen L, Kerr M A. Unaggregated serum IgA binds to neutrophil FcαR at physiological concentrations and is endocytosed but cross-linking is necessary to elicit a respiratory burst. J Leukocyte Biol. 56:481-487, 1994.

Symersky J, Novak J, McPherson D T, DeLucas L, Mestecky J. Expression of the recombinant human immunoglobulin J chain in Escherichia coli. Mol Immunol. 37:133-40, 2000.

Teitsson I, Withrington R h, Seifert M H, Valdimarsson H. Prospective study of early rheumatoid arthritis. I. Prognostic value of IgA rheumatoid factor. Ann. Rheum. Dis. 43:673-8, 1984.

Tuffaha A, Gern J E, Lemanske R F Jr. The role of respiratory viruses in acute and chronic asthma. Clin Chest Med. 21:289-300, 2000.

Valerius T, Stockmeyer B, van Spriel A B, Graziano R F, van den Herik-Oudijk I E, Repp R, Deo Y M, Lund J, Kalden J R, Gramatzki M, van de Winkel J G. FcalphaRI (CD89) as a novel trigger molecule for bispecific antibody therapy. Blood. 90:4485-92, 1997.

van Egmond M, van Vuuren A J, Morton H C, van Spriel A B, Shen L, Hofhuis F M, Saito T, Mayadas T N, Verbeek J S, van de Winkel J G. Human immunoglobulin A receptor (FcalphaRI, CD89) function in transgenic mice requires both FcR gamma chain and CR3 (CD11b/CD18). Blood. 93:4387-94, 1999.

Yeaman G R, Kerr M A. Opsonization of yeast by human serum IgA anti-mannan antibodies and phagocytosis by human polymorphonuclear leucocytes. Clin. Exp. Immunol. 68:200-8, 1987.

1. A method for modulating the inflammatory response of a mesenchymal cell comprising administering an effective amount of an agent that can modulate an IgA receptor on a mesenchymal cell to a cell or animal in need thereof

2. A method according to claim 1 to inhibit the inflammatory responses of a mesenchymal cell.

3. A method according to claim 2 to treat an inflammatory condition caused by an IgA binding to an IgA receptor on a mesenchymal cell.

4. A method according to claim 3 wherein the. inflammatory condition is arthritis.

5. A method according to claim 4 wherein the arthritides is selected from rheumatoid arthritis, osteoarthritis or a spondyloarthropathy.

6. A method according to claim 3 wherein the inflammatory condition is selected from Crohn's disease, ulcerative colitis, Behcet's disease, Sjogren's disease and a vasculitis.

7. A method according to claim 3 wherein the condition is asthma, chronic bronchitis, acute bronchitis, bronchial hyperreactivity, chronic obstructive pulmonary disease, emphysema, interstitial lung disease, bronchiectasis or airway remodelling.

8. A method for modulating cytosolic calcium signalling in a mesenchymal cell comprising administering an effective amount of an agent that can modulate an IgA receptor on a mesenchymal cell to a cell or animal in need thereof.

9. A method according to claim 8 comprising administering an effective amount of an IgA receptor antagonist to prevent or inhibit intracellular calcium signalling in a mesenchymal cell.

10. A method for inhibiting the contraction of a mesenchymal cell comprising administering use of an effective amount of an IgA receptor antagonist to a cell or animal in need thereof.

11. A method for inhibiting the production of inflammatory mediators or growth factors comprising administering an effective amount of an IgA receptor antagonist to a cell or animal in need thereof.

12. A method according to claim 1 wherein the IgA receptor is pIgR or FcαR.

13. A method according to claim 2 wherein the IgA receptor antagonist inhibits the binding of pIgA to pIgR.

14. A method according to claim 2 wherein the IgA receptor antagonist inhibits the binding of pIgA to FcαR.

15. A method according to claim 2 wherein the IgA receptor antagonist is a scFv that binds pIgR or FcαR.

16. A method according 1 wherein the mesenchymal cell is a smooth muscle cell.

17. A method according to claim 16 wherein the cell is an airway smooth muscle cell.

18. A method according to claim 1 wherein the mesenchymal cell is a fibroblast.

19. A method according to claim 18 wherein the cell is a synovial fibroblast.

20. A method of delivering a substance to a mesenchymal cell comprising administering to an animal or cell in need thereof an effective amount of a conjugate comprising the substance coupled to an IgA receptor ligand.

21. A method according to claim 20 wherein the IgA receptor is pIgR or FcαR.

22. A method according to claim 20 wherein the mesenchymal cell is a fibroblast or smooth muscle cell.

23. A method of detecting a condition associated with the activation of a mesenchymal IgA receptor on a mesenchymal cell comprising assaying a tissue sample or cells from the sample for (a) a nucleic acid molecule encoding an IgA receptor or a fragment thereof or (b) an IgA receptor or a fragment thereof.

24. A method according to claim 23 wherein the IgA receptor is pIgR or FcαR.

25. A method according to claim 23 wherein the condition is an inflammatory condition selected from arthritides, including rheumatoid arthritis, osteoarthritis, spondyloarthropathies, Crohn's disease, ulcerative colitis, Behcet's disease, Sjogren's disease and vasculitides.

26. A method according to claim 23 wherein the condition is asthma, chronic Dronchitis, acute bronchitis, bronchial hyperreactivity, chronic obstructive pulmonary disease, emphysema, interstitial lung disease, bronchiectasis or airway remodelling.

27. A method of detecting IgA mediated bronchial hyperreactivity comprising:
(a) administering an IgA receptor agonist to a patient; and
(b) detecting bronchoconstriction in the patient wherein an increase in bronchoconstriction as compared to a control indicates that the patient has IgA-mediated hyperreactivity.

28. A method according to claim 27 wherein bronchoconstriction is measured by listening for wheezing on chest auscultation.

29. A method according to claim 27 wherein bronchoconstriction is measured by measuring a reduced forced expiratory volume at 1 second (FEV1).

30. A method of detecting IgA-mediated bronchial hyperreactivity comprising:
(a) administering an IgA-receptor agonist to a patient and detecting bronchoconstriction; and
(b) administering an IgA receptor agonist followed by a non-specific bronchoconstricting agent to the patient and detecting bronchoconstriction at a lower dose than when the nonspecific agent is administered alone wherein bronchoconstriction in step (a) and/or bronchoconstriction induced at a lower dose of the nonspecific agent administered without the IgA receptor agonist in step (b) would indicate that the patient has IgA-mediated bronchial hyperreactivity.

31. A method according to claim 30 wherein the non-specific bronchoconstricting agent is methacholine or histamine.

32. A method according to claim 30 wherein bronchoconstriction is detected with a pulmonary function test such as clinical spirometry [=measurement of FEV1 and FVC].
